(19) United States
(12) Patent Application Publication
(10) Pub. No.: US 2026/0069622 A1
(43) Pub. Date: Mar. 12, 2026

(54) COMPOSITIONS AND METHODS FOR TREATING VASCULAR MALFORMATION AND RELATED CONDITIONS

(71) Applicants: THE JOHNS HOPKINS UNIVERSITY, Baltimore, MD (US); DUKE UNIVERSITY, Durham, NC (US); KENNEDY KRIEGER INSTITUTE, INC., Baltimore, MD (US)

(72) Inventors: Anne Comi, Baltimore, MD (US); Jonathan Pevsner, Baltimore, MD (US); Zhenhua Huang, Ellicott City, MD (US); Doug Marchuk, Chapel Hill, NC (US)

(21) Appl. No.: 17/935,236

(22) Filed: Sep. 26, 2022

Related U.S. Application Data
(63) Continuation of application No. 15/750,674, now Pat. No. 11,452,737.
(60) Provisional application No. 62/201,859, filed on Aug. 6, 2015.
(60) Provisional application No. 62/302,035, filed on Mar. 1, 2016.

(51) Int. Cl. A61K 31/7076; A61P 17/00; A61P 35/00; C12Q 1/68; C12Q 1/6883

(52) U.S. Cl. CPC A61K 31/7076; A61P 17/00; A61P 35/00; C12Q 1/68; C12Q 1/6883; C12Q 2600/106; C12Q 2600/112; C12Q 2600/118; C12Q 2600/142; C12Q 2600/156

(57) ABSTRACT

In one aspect, the present invention features a method of inhibiting proliferation and/or reducing survival of a cell comprising a GNAQ polynucleotide or polypeptide having a R183Q or Q209L mutation, comprising contacting the cell with puromycin or a puromycin analog, thereby inhibiting proliferation and/or reducing survival of the cell. In another aspect, a method of treating a vascular malformation or related condition in a subject, comprising administering to the subject an effective amount of puromycin or a puromycin analog is featured. In another aspect, the present invention features a method of identifying a candidate agent that modulates a GNAQ R183Q or Q209L mutation-associated disease, comprising contacting a cell comprising a GNAQ polynucleotide or polypeptide having a R183Q or Q209L mutation with puromycin and a candidate agent and comparing viability of the contacted cell with a reference level of viability, wherein an alteration in viability indicates that the candidate agent modulates the GNAQ R183Q or Q209L mutation-associated disease.

### REFERENCE TO RELATED APPLICATIONS

This application is a continuation of U.S. patent application Ser. No. 15/750,674, filed on Feb. 6, 2018, now U.S. Pat. No. 11,452,737, issued on Sep. 27, 2022, which is a 35 U.S.C. § 371 U.S. national entry of International Application PCT/US2016/044442, filed on Jul. 28, 2016, which claims the benefit of U.S. Provisional Patent Application No. 62/201,859, filed on Aug. 6, 2015, and U.S. Provisional Patent Application No. 62/302,035, filed on Mar. 1, 2016, each of which are hereby incorporated by reference for all purposes as if fully set forth herein.

### STATEMENT OF GOVERNMENT INTEREST

This invention was made with government support under grant no. UCSF 5842SC U54 awarded by the National Institutes of Health. The government has certain rights in the invention.

### REFERENCE TO AN ELECTRONIC SEQUENCE LISTING

The text of the computer readable sequence listing filed herewith, titled “P13712-05”, created Apr. 19, 2023, having a file seize of 46,685 bytes, is hereby incorporated by reference in its entirety.

### BACKGROUND OF THE INVENTION

Capillary malformations (port-wine birthmarks) occur in about 1 in 300 live births. Sturge-Weber syndrome is the same vascular malformation involving the brain, skin and eye. Patients with Sturge-Weber syndrome are at risk of developing glaucoma, seizures, strokes, and neurological impairment. Current treatments for vascular malformation are primarily symptomatic and inadequate. Thus, new methods of treating vascular malformation are urgently required.

### SUMMARY OF THE INVENTION

As described below, the present invention features compositions and methods for treating vascular malformation and related conditions.

In one aspect, the present invention provides a method of inhibiting proliferation and/or reducing survival of a cell containing a GNAQ polynucleotide or polypeptide having a R183Q or Q209L mutation, the method containing the step of contacting the cell with puromycin or a puromycin analog, thereby inhibiting proliferation and/or reducing survival of the cell.

In another aspect, the present invention provides a method of reducing a vascular malformation in a subject, the method containing the step of administering to the subject an effective amount of puromycin or a puromycin analog.

In another aspect, the present invention provides a method of inhibiting progression of a vascular malformation in a subject, the method containing the step of administering to the subject an effective amount of puromycin or a puromycin analog.

In another aspect, the present invention provides a method of reducing appearance of a birthmark in a subject, the method containing the step of administering to the subject an effective amount of puromycin or a puromycin analog.

In another aspect, the present invention provides a method of treating a vascular malformation or related condition in a subject, the method containing the step of administering to the subject an effective amount of puromycin or puromycin analog.

In another aspect, the present invention provides a method of treating a uveal melanoma in a subject, the method containing the step of administering to the subject an effective amount of puromycin or puromycin analog.

In another aspect, the present invention provides a method of treating a disease associated with a R183Q or Q209L mutation in a subject, the method containing the step of administering to the subject an effective amount of puromycin or puromycin analog.

In various embodiments of any aspect delineated herein, the method contains the step of administering puromycin or puromycin analogue in addition to laser treatment to the subject. In various embodiments of any aspect delineated herein, the vascular malformation or related condition is a capillary malformation, vascular malformation in the brain, vascular malformation in the eye, or Sturge-Weber syndrome. In various embodiments of any aspect delineated herein, the puromycin or puromycin analog is administered topically, orally, by injection, or by ocular administration.

In various embodiments of any aspect delineated herein, the subject comprises a GNAQ polynucleotide or polypeptide having a R183Q or Q209L mutation. In various embodiments of any aspect delineated herein, the subject is a human.

In another aspect, the present invention provides a composition comprising a puromycin or puromycin analog formulated for topical administration, ocular administration, oral administration, or administration by injection. In various embodiments, the method according to any other aspect delineated herein contains the step of administering to the subject an effective amount of the composition of any other aspect delineated herein.

In another aspect, the present invention provides a transfected human embryonic kidney (HEK) or endothelial cell containing an isolated polynucleotide encoding a GNAQ polypeptide containing a R183Q or Q209L mutation. In various embodiments of any aspect delineated herein, the cell further contains an isolated polynucleotide encoding a puromycin resistance polypeptide. In various embodiments, the cell is HEK293, HEK293T, EA.926, EA.hy 926, or HUVEC. In various embodiments of any aspect delineated herein, the isolated polynucleotide encoding a GNAQ polypeptide containing a R183Q or Q209L mutation is in a lentivirus plasmid. In various embodiments, the cell is stably transfected or transiently transfected with the isolated polynucleotide encoding a GNAQ polypeptide containing a R183Q or Q209 Lmutation. In another aspect, the present invention provides a method of identifying a candidate agent that modulates a GNAQ R183Q or Q209L mutation-associated disease, the method containing the steps of (a) contacting a cell comprising a GNAQ polynucleotide or polypeptide having a R183Q or Q209L mutation with puromycin and a candidate agent; and (b) comparing viability of the contacted cell with a reference level of viability, wherein an alteration in viability indicates that the candidate agent modulates the GNAQ R183Q or Q209L mutation-associated disease.

In another aspect, the present invention provides a method of identifying a candidate agent that modulates a vascular malformation or related condition, the method containing the steps of (a) contacting a cell comprising a GNAQ polynucleotide or polypeptide having a R183Q or Q209 Lmutation with puromycin and a candidate agent; and (b) comparing viability of the contacted cell with a reference level of viability, where an alteration in viability indicates that the candidate agent modulates a vascular malformation or related condition. In various embodiments, the cell is a cell according to any aspect delineated herein.

In various embodiments of any aspect delineated herein, the alteration in viability is positive or negative. In various embodiments of any aspect delineated herein, the GNAQ R183Q or Q209L mutation-associated disease or the vascular malformation or related condition is a capillary malformation, vascular malformation in the brain, vascular malformation in the eye, or Sturge-Weber syndrome.

In another aspect, the invention provides a method of identifying an agent that modulates a vascular malformation or related condition, the method containing the steps of (a) contacting a cell with a candidate agent; and (b) measuring a level or activity of a ID3, TSC22D3, or TEAD3 polynucleotide or polypeptide, where an alteration in the level or activity of the ID3, TSC22D3, or TEAD3 polynucleotide or polypeptide, indicates that the candidate agent modulates a vascular malformation or related condition.

In various embodiments of any one of the aspects delineated herein, the cell contains a GNAQ polynucleotide or polypeptide containing a R183Q or Q209L mutation. In various embodiments, the cell is a cell of any one of the aspects delineated herein. In some embodiments, the alteration is an increase or decrease in the level or activity of the ID3, TSC22D3, or TEAD3 polynucleotide or polypeptide.

Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels. “

By “analog” or “analogue” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid. By “puromycin analog” a molecule that is not identical, but has analogous functional or structural features to puromycin. Puromycin analogs include, without limitation, compounds derived from puromycin and having an inhibitory effect on protein translation. Examples of puromycin analogs include, without limitation, N⁶-bis-demethylpuromycin, (2S)-N-{(1R,2S,4S,5S)-4-[6-(Dimethylamino) purin-9-yl]-1-(hydroxymethyl) bicyclo[3.1.0]hex-2-yl}-2-amino-3-(4-methoxyphenyl) propanamide, (25)-N-{(15,3S,4S,5S)-1-[6-(dimethylamino) purin-9-yl]-4-(hydroxymethyl) bicyclo[3.1.0]hex-3-yl}-3-(4-methoxy-phenyl) propanamide, (2S)—N-[(1R,2S,45,5S)-4-(6-aminopurin-9-yl)-1-(hydroxymethyl) bicyclo[3.1.0]hex-2-yl]-2-amino-3-(4-methoxyphenyl) propanamide, and (25)-N-[(1S,3S,4S,5S)-1-(6-aminopurin-9-yl)-4-(hydroxy-methyl) bicyclo[3.1.0]hexan-3-yl]-2-amino-3-(4-methoxy-phenyl) propanamide.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “disease” is meant any condition or disorder that damages or interferes with the normal function of a cell, tissue, or organ. By “disease associated with a GNAQ R183Q or Q209 Lmutation” or “GNAQ R183Q or Q209L mutation-associated disease” is meant a disease caused by or associated with a R183Q or Q209 Lmutation in a GNAQ polynucleotide or polypeptide. Examples of diseases associated with a GNAQ R183Q or Q209L mutation include vascular malformation, capillary malformation, vascular malformation in the brain, vascular malformation in the eye, Sturge-Weber syndrome, and Q209L or R183Q uveal melanoma.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The invention provides a number of targets that are useful for the development of highly specific drugs to treat or a disorder characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “GNAQ polypeptide” is meant a polypeptide or fragment thereof having at least about 85% or greater amino acid identity to the amino acid sequence provided at NCBI Accession No. NP_002063 and having GNAQ or Gaq biological activity. Exemplary GNAQ or Gaq biological activities include activation of phospholipase C (PLC) and modulation of signaling pathways in a cell. An exemplary GNAQ polypeptide sequence (SEQ ID. NO: 1) is provided below:

| | |
|---|---|
| 1 | mtlesimacc lseeakearr indeierqlr rdkrdarrel kllllgtges gkstfikqmr |
| 61 | iihgsgysde dkrgftklvy qniftamqam iramdtlkip ykyehnkaha qlvrevdvek |
| 121 | vsafenpyvd aikslwndpg iqecydrrre yqlsdstkyy lndldrvadp aylptqqdvl |
| 181 | rvrvpttgii eypfdlqsvi frmvdvggqr serrkwihcf envtsimflv alseydqvlv |
| 241 | esdnenrmee skalfrtiit ypwfqnssvi lflnkkdlle ekimyshlvd yfpeydgpqr |
| 301 | daqaarefil kmfvdlnpds dkiiyshftc atdtenirfv faavkdtilq lnlkeynlv |

By “GNAQ polynucleotide” is meant a polynucleotide encoding a GNAQ polypeptide. An exemplary GNAQ polynucleotide sequence is provided at NCBI Accession No. NM 002072 (SEQ ID NO: 2). SEQ ID NO:2 is provided below:

| | |
|---|---|
| 1 | agactatccg ctcccaccgc gcccccggcc cacctggtgg ccccggccct ggccgccgcc |
| 61 | cccgcggcgg ttcccggagc tcgtcccgga cgcgcgcccg ggcggcgggg gctcggcggc |
| 121 | caccgctgcc tcgggggagc gagggcggga gggtgtgtgt gcgcgctgtg agcagggggt |
| 181 | gccggcgggg ctgcagcgga ggcactttgg aagaatgact ctggagtcca tcatggcgtg |
| 241 | ctgcctgagc gaggaggcca aggaagcccg gcggatcaac gacgagatcg agcggcagct |
| 301 | ccgcagggac aagcgggacg cccgccggga gctcaagctg ctgctgctcg ggacaggaga |
| 361 | gagtggcaag agtacgttta tcaagcagat gagaatcatc catgggtcag gatactctga |
| 421 | tgaagataaa aggggcttca ccaagctggt gtatcagaac atcttcacgg ccatgcaggc |
| 481 | catgatcaga gccatggaca cactcaagat cccatacaag tatgagcaca ataaggctca |
| 541 | tgcacaatta gttcgagaag ttgatgtgga gaaggtgtct gcttttgaga atccatatgt |
| 601 | agatgcaata aagagtttat ggaatgatcc tggaatccag gaatgctatg atagacgacg |
| 661 | agaatatcaa ttatctgact ctaccaaata ctatcttaat gacttggacc gcgtagctga |
| 721 | ccctgcctac ctgcctacgc aacaagatgt gcttagagtt cgagtcccca ccacagggat |
| 781 | catcgaatac ccctttgact tacaaagtgt cattttcaga atggtcgatg tagggggcca |
| 841 | aaggtcagag agaagaaaat ggatacactg ctttgaaaat gtcacctcta tcatgtttct |
| 901 | agtagcgctt agtgaatatg atcaagttct cgtggagtca gacaatgaga accgaatgga |
| 961 | ggaaagcaag gctctcttta gaacaattat cacatacccc tggttccaga actcctcggt |
| 1021 | tattctgttc ttaaacaaga aagatcttct agaggagaaa atcatgtatt cccatctagt |
| 1081 | cgactacttc ccagaatatg atggacccca gagagatgcc caggcagccc gagaattcat |
| 1141 | tctgaagatg ttcgtggacc tgaacccaga cagtgacaaa attatctact cccacttcac |
| 1201 | gtgcgccaca gacaccgaga atatccgctt tgtctttgct gccgtcaagg acaccatcct |
| 1261 | ccagttgaac ctgaaggagt acaatctggt ctaattgtgc ctcctagaca cccgccctgc |
| 1321 | ccttccctgg tgggctattg aagatacaca agagggactg tatttctgtg gaaaacaatt |
| 1381 | tgcataatac taatttattg ccgtcctgga ctctgtgtga gcgtgtccac agagtttgta |
| 1441 | gtaaatatta tgattttatt taaactattc agaggaaaaa cagaggatgc tgaagtacag |
| 1501 | tcccagcaca tttcctctct atcttttttt taggcaaaac cttgtgactc agtgtatttt |
| 1561 | aaattctcag tcatgcactc acaaagataa gacttgtttc tttctgtctc tctctctttt |
| 1621 | tcttttctat ggagcaaaac aaagctgatt tccctttttt cttcccccgc taattcatac |
| 1681 | ctccctcctg atgtttttcc caggttacaa tggcctttat cctagttcca ttcttggtca |
| 1741 | agtttttctc tcaaatgata cagtcaggac acatcgttcg atttaagcca tcatcagctt |
| 1801 | aatttaagtt tgtagttttt gctgaaggat tatatgtatt aatacttacg gttttaaatg |
| 1861 | tgttgctttg gatacacaca tagtttcttt tttaatagaa tatactgtct tgtctcactt |
| 1921 | tggactggga cagtggatgc ccatctaaaa gttaagtgtc atttctttta gatgtttacc |
| 1981 | ttcagccata gcttgattgc tcagagaaat atgcagaagg caggatcaaa gacacacagg |
| 2041 | agtcctttct tttgaaatgc cacgtgccat tgtctttcct cccttctttg cttctttttc |
| 2101 | ttaccctctc tttcaattgc agatgccaaa aaagatgcca acagacacta cattacccta |
| 2161 | atggctgcta cccagaacct ttttataggt tgttcttaat ttttttgttg ttgttgttca |
| 2221 | agcttttcct ttcttttttt tcttggtgtt tgggccacga ttttaaaatg acttttatta |
| 2281 | tgggtatgtg ttgccaaagc tggctttttg tcaaataaaa tgaatacgaa cttaaaaaat |
| 2341 | aaaagctggt atcttaaaat gtaagagagt aagactgtga agcctaaaat gactggctga |
| 2401 | gaatgaacca gaaatgccat ttgccaaaca gttgtaacta gaaatttgat tctcacggtc |
| 2461 | cattcttttc tttgtcctta agatgacatt gttagtgttc acgtcccatg ttcagtgtcc |
| 2521 | aaaccggcaa tgtaaaaagt atcctgtgtg gtttaacagg aaatctgttt atgtctcttt |
| 2581 | atttgaaacc agttttactc tcagtggttc tttaagttca atgaagtctg ccaggaacat |
| 2641 | tggttggtag tattattccg acacctttaa tttccaaaat ctgaagttcc tgctagttta |
| 2701 | ccaccttcat gatcttcttg aactggtaac tgattaggtt gaacttatgg aagatttgtg |
| 2761 | gacttaactc aaaagtaacc tctcagtgtt ctatagaaca tgtatttgtg taactgaacc |
| 2821 | taccaggaga aatgtttgga attctatatg tgcaattttt caacaaatgc aaaaaaaata |
| 2881 | cagcacatgt attgacaagc ttctgtcaag cagcttgagt tgaaatttga tttaagaaaa |
| 2941 | taaatcatga ttgttcaaag ctgctgggac gttagaatta ggccatgata ctggtctcat |
| 3001 | tttaactaca gtggtatttg gcactagtgt aaacttccat ataaatcact cttttggaac |
| 3061 | aacaaagggg gagggagaaa aatcacggcc tgttaaatga gtaccaaagc cgcccaacag |
| 3121 | taatgagatg ttctcatcct tgattctccc agcctcaaac aacacagctt actttttttt |
| 3181 | tcccttgctc agaaagtacc tgtaatttaa caaacagact gcctgtaggt atagtgcaat |
| 3241 | tacaaatgct ctaatcattg tacatacatc tctcttgata ttgcagcatc catactggct |
| 3301 | ttgtaatcat taattttttg gcagattgaa tgtgctgtat tgatatgtat ctatgtaatt |
| 3361 | gtattgtatg tctatagcta attcacgttt tgaataatgt tattttattt acttttttaa |
| 3421 | gagaggagaa tgtaaatttg tcagtttatt tctgactagg gatattttct ttccatttag |
| 3481 | aaaagaagaa aaaaaaaaaa ccttactgtc atacagagcg gtactagcgt cgtgctgtat |
| 3541 | aaaatcattt gcacattcct gagtagaggt atactgatta taagacccaa aggtaatttc |
| 3601 | atagcaaaat acataaaatc agtcggagct tttatacaaa catggaaacc aactttgtag |
| 3661 | aacttttgcc atttgatcta ggattggaat atgagctttt atacaattca tattcttatt |
| 3721 | tggcaaatgc acagtttagt attacctctc tgatggcctt tactagaaag gcagttttag |
| 3781 | aagctattgt gatccactaa ggaaatgttt taacagctag agaccactgc ttgcctgaaa |
| 3841 | gggcgttctt aaatttggtg cagcaaaaaa aaaaaaaaaa aaaaaaaaaa ttaaacaaca |
| 3901 | acatttgaag gcctacagtg tgtatagaga aaacctcatc acaagatcat aagtgttaca |
| 3961 | gttttaggga atcaagatat tctatttaat agagctatag taaatgtagt caattaaacc |
| 4021 | tgatctcaaa gcttgaagaa gctgagcaaa acagggaaag attgttatat ttgtctttat |
| 4081 | gaaattggga tggaatttgc tatgcagaat tgaggtttgt ggcttcgctg ttcctgtagg |
| 4141 | gtgcatgaca agatcccttc tcttgagaaa ggaaaaaatt gatcacccta gcagcagtga |
| 4201 | tgcatagaaa cctaatttta gccacaccag tcaatcgaag ctaaaggatt ttcttttttg |
| 4261 | tttcttcggg gttttattga aggggctagg ggcgggacgg gattcttttc agttttgtat |
| 4321 | aaaaacaaag tttactcatg ctttatatta tattgtgatt gcaagcgtta taagcgtgtg |
| 4381 | ccactggcct cctattgttg atgcttaggt aatggaggcc tgtggtgagt tttatggtga |
| 4441 | cttgggcatg tcttattcaa aaacaaaaac ataaaacaca gaaacctttc ttcagcatac |
| 4501 | caaggcaagc agccatttca tgactcactt aacacattgc agtgtaccag tttacagatg |
| 4561 | atttttccct ttttgcgtga catggcagtt ctaaccccca gagaattcct tatttgtaaa |
| 4621 | ttggaagttt ctactatgcc ttacagagct taaattcaga agtttgtgcc tcatatctga |
| 4681 | aacaaaggga aataacacac ccattcaaaa gtaaataaat ctcctagaag tttttgtttt |
| 4741 | taacatttcc atataaagag ctctgttgaa tgtcatgaat agactggaaa aaaaaatttt |
| 4801 | aagaacctgc atatgttgtt tactagcaga tgacaactac aaaaggaatc tgaagaacac |
| 4861 | gtaaaacttg tatttttttt tttttggtag attaactagc aggcctattt taaaaaggta |
| 4921 | attcagctaa agggcaattt acttttttgt acttcagact atcttgattg tcaaagtgta |
| 4981 | cgaactgtaa ttttaaaatt tatactgcca catgattgta aattttagtt gtcttaagtt |
| 5041 | aggaattggt gaaaagctat ttatgctgga tttgggtcaa aatgacttat ttgcaaaaaa |
| 5101 | ataaataatg ggaagaaagg gctgtataat gaaatactgc aagactcaca tattggttgg |
| 5161 | aaatttccct caaatcacct accgattacc cttgatttcc ctttgttttc agtttctcaa |
| 5221 | aacgaatgaa atgaaatata gcagaatgtt aacccatata aaaataaagt gtacccaaat |
| 5281 | attgtaatgt atattgctgc tcttcttcaa attaaataag ggtttaaaac cacttaattg |
| 5341 | gtaatcaaca tctcaattga tacaaataag gtgtgcttgg tatacattaa tattttcttc |
| 5401 | caaagatata tctttggtta gaaacacaaa aaaataaaac tagtaatatt gtatgtttat |
| 5461 | ctatctctac atatttccag catatgtagc gttaatagat ctgtcctggt aactgtgtct |
| 5521 | ttgggatttc attttggttc catcaaatta ggaaaagaaa tggcttagtt gtatatgatt |
| 5581 | agctagagat ttttggagcc agacacctgc tgtttagtag ataacttagt acagacccta |
| 5641 | aacttgtcat ttgtttttct cacagaatag ccatttcctg ctgtcttccc aatgatcact |
| 5701 | gccctttcaa taacactctt gcctctagaa tcatatgttc aaagtatgaa tacacaccta |
| 5761 | gcacatagta ggtgctcaaa tattaatttc ctccttgcct tccttatcta ccctgtgtcc |
| 5821 | tccatttccc cgtatgattc caacccaata tagcaaatga catttacatg ttatgaaaac |
| 5881 | atctattggg taaaatcaga tcttggataa agaaattctg acttttatat aagcttttgg |
| 5941 | tagacagaaa aaacagaaag gtattcgttg gtagaacatt tttaagttca ggaaagaaag |
| 6001 | ctggaataat actacgtaac tttgtccagg ttactttgac tgaaacacgt ttttggtgga |
| 6061 | tttcttttcc tcaaagaact ctctaaatgc aactccttgc tggattcctc acccatcatc |
| 6121 | ctgttggaaa cccttactag acctatgtat ttagggagtt ttgtcagaaa acatttttaa |
| 6181 | cttgcagtat ttaaaagaat atttactgtt cctaaaatgt cattcaaatg catgtactgt |
| 6241 | ctattgtttg gggatgggaa ctagttttgc aaaaaacacc taatgttgta taataatgcc |
| 6301 | ccaatgatct tgctggttaa aaatacagta tttttggcca taa |

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “ID3 polypeptide” is meant a polypeptide or fragment thereof having at least about 85% or greater amino acid identity to the amino acid sequence provided at NCBI Accession No. NP_002158.3 and having ID3 polypeptide biological activity. Exemplary ID3 biological activities include forming heterodimer with helix-loop-helix (HLH) proteins and inhibition of DNA binding activity and transcriptional activity of basic helix-loop-helix transcription factors.

An exemplary ID3 polypeptide sequence is provided below (NP_002158.3) (SEQ ID NO:3):

| | |
|---|---|
| 1 | mkalspvrgc yeavcclser slaiargrgk gpaaeeplsl lddmnhcysr lrelvpgvpr |
| 61 | gtqlsqveil grvidyildl qvvlaepapg ppdgphlpiq taeltpelvi sndkrsfch |

By “ID3 polynucleotide” is meant a polynucleotide encoding a ID3 polypeptide. An exemplary ID3 polynucleotide sequence is provided at NCBI Accession No. NM_002167.4 (SEQ ID NO:4). SEQ ID NO:4 is provided below:

| | |
|---|---|
| 1 | gatctggggt gctgccagga aaaagcaaat tctggaagtt aatggttttg agtgattttt |
| 61 | aaatccttgc tggcggagag gcccgcctct ccccggtatc agcgcttcct cattctttga |
| 121 | atccgcggct ccgcggtctt cggcgtcaga ccagccggag gaagcctgtt tgcaatttaa |
| 181 | gcgggctgtg aacgcccagg gccggcgggg gcagggccga ggcgggccat tttgaataaa |
| 241 | gaggcgtgcc ttccaggcag gctctataag tgaccgccgc ggcgagcgtg cgcgcgttgc |
| 301 | aggtcactgt agcgggactt cttttggttt tctttctctt tggggcacct ctggactcac |
| 361 | tccccagcat gaaggcgctg agcccggtgc gcggctgcta cgaggcggtg tgctgcctgt |
| 421 | cggaacgcag tctggccatc gcccggggcc gagggaaggg cccggcagct gaggagccgc |
| 481 | tgagcttgct ggacgacatg aaccactgct actcccgcct gcgggaactg gtacccggag |
| 541 | tcccgagagg cactcagctt agccaggtgg aaatcctaca gcgcgtcatc gactacattc |
| 601 | tcgacctgca ggtagtcctg gccgagccag cccctggacc ccctgatggc ccccaccttc |
| 661 | ccatccagac agccgagctc actccggaac ttgtcatctc caacgacaaa aggagctttt |
| 721 | gccactgact cggccgtgtc ctgacacctc cagaacgcag gtgctggcgc ccgttctgcc |
| 781 | tgggaccccg ggaacctctc ctgccggaag ccggacggca gggatgggcc ccaacttcgc |
| 841 | cctgcccact tgacttcacc aaatcccttc ctggagacta aacctggtgc tcaggagcga |
| 901 | aggactgtga acttgtggcc tgaagagcca gagctagctc tggccaccag ctgggcgacg |
| 961 | tcaccctgct cccaccccac ccccaagttc taaggtctct tcagagcgtg gaggtgtgga |
| 1021 | aggagtggct gctctccaaa ctatgccaag gcggcggcag agctggtctt ctggtctcct |
| 1081 | tggagaaagg ttctgttgcc ctgatttatg aactctataa tagagtatat aggttttgta |
| 1141 | ccttttttac aggaaggtga ctttctgtaa caatgcgatg tatattaaac tttttataaa |
| 1201 | agttaacatt ttgcataata aacgattttt aaacacttga aaaaaaaaaa aa |

By “increasing cell survival” is meant positively altering cell viability. By “reducing cell survival” is meant negatively altering cell viability.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder or that is associated with sensitivity or resistance to an agent. For example, a R183Q mutation in a GNAQ polynucleotide or polypeptide is a marker for Sturge-Weber syndrome, capillary malformation, and uveal melanoma. The R183Q mutation in a GNAQ polynucleotide or polypeptide is also a marker for puromycin sensitivity of a cell.

As used herein, “obtaining” as in “obtaining an agent” includes synthesizing, purchasing, or otherwise acquiring the agent.

As used herein, a “puromycin resistance polypeptide” is a polypeptide that confers resistance to puromycin in cells. Exemplary puromycin resistance polypeptides include, without limitation, a Pac (puromycin N-acetyltransferase) polypeptide. An exemplary sequence of a puromycin resistance polypeptide is provided at UniProtKB Accession No. P13249 (SEQ ID NO: 5). SEQ ID NO:5 is provided below.

```
            10 20 30 40 50
MTEYKPTVRL ATRDDVPRAV RTLAAAFADY aATRHTVDPD RHIERVTELQ
                60 70 80 90 100
ELFLTRVGLD IGKVWVADDG AAVAVWTTPE SVEAGAVFAE IGPRMAELSG
              110 120 130 140 150
SRLAAQQQME GLLAPHRPKE PAWFLATVGV SPDHQGKGLG SAVVLPGVEA
              160 170 180 190
AERAGVPAFL ETSAPRNLPF YERLGFTVTA DVEVPEGPRT WCMTRKPGA
```

“Primer set” means a set of oligonucleotides that may be used, for example, for PCR. A primer set would consist of at least 2, 4, 6, 8, 10, 12, 14, 16, 18, 20, 30, 40, 50, 60, 80, 100, 200, 250, 300, 400, 500, 600, or more primers.

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “specifically binds” is meant a compound or antibody that recognizes and binds a polypeptide of the invention, but which does not substantially recognize and bind other molecules in a sample, for example, a biological sample, which naturally includes a polypeptide of the invention.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100.mu.g/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1%

SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

By “subject” is meant a mammal, including, but not limited to, a human or non-human mammal, such as a murine, bovine, equine, canine, ovine, or feline.

By “TEAD3 polypeptide” is meant a polypeptide or fragment thereof having at least about 85% or greater amino acid identity to the amino acid sequence provided at NCBI Accession No. NP_003205.2, and having TEAD3 polypeptide biological activity. Exemplary TSC22D3 biological activities include transcription factor and DNA-binding activity. An exemplary TEAD3 polypeptide sequence is provided below (NP_003205.2) (SEQ ID NO:6):

| | |
|---|---|
| 1 | masnswnass spgearedgp egldkgldnd aegvwspdie qsfqealaiy ppcgrrkiil |
| 61 | sdegkmygrn eliaryiklr tgktrtrkqv sshiqvlark kvreyqvgik amnldqvskd |
| 121 | kalqsmasms saqivsasvl qnkfsppspl pqavfstssr fwssppllgq qpgpsqdikp |
| 181 | faqpaypiqp plpptlssye plaplpsaaa svpvwqdrti assrlrlley safmevqrdp |
| 241 | dtyskhlfvh igqtnpafsd ppleavdvrq iydkfpekkg glkelyekgp pnafflvkfw |
| 301 | adlnstiqeg pgafygvssq yssadsmtis vstkvcsfgk qvvekvetey arlengrfvy |
| 361 | rihrspmcey minfihklkh lpekymmnsv lenftilqvv tsrdsqetll viafvfevst |
| 421 | sehgaqhhvy klvkd |

By “TEAD3 polynucleotide” is meant a polynucleotide encoding a TEAD3 polypeptide. An exemplary ID3 polynucleotide sequence is provided at NCBI Accession No. NM_003214.3 (SEQ ID NO:7). SEQ ID NO:7 is provided below:

| | |
|---|---|
| 1 | tcctcaacac aaactttccg tcccgctcgc tccctcctcc gcgctcggcg cctcccgctc |
| 61 | cagcccggct cattccgcac attccggcca gccccctccc cacgaccccc cttccccggc |
| 121 | cccccttgcg gctccctcgg gcccggcgga gcggcccggc cggagcgccc ccgcgagctc |
| 181 | ggaccaggct cagccgccca gtgggctcag gcccagagcc cagagcaacc agcacaatag |
| 241 | cgtccaacag ctggaacgcc agcagcagcc ccggggaggc ccgggaggat gggcccgagg |
| 301 | gcctggacaa ggggctggac aacgatgcgg agggcgtgtg gagcccggac atcgagcaga |
| 361 | gcttccagga ggccctggcc atctacccgc cctgcggccg gcggaagatc atcctgtcag |
| 421 | acgagggcaa gatgtacggc cgaaatgagt tgattgcacg ctatattaaa ctgaggacgg |
| 481 | ggaagactcg gacgagaaaa caggtgtcca gccacataca ggttctagct cggaagaagg |
| 541 | tgcgggagta ccaggttggc atcaaggcca tgaacctgga ccaggtctcc aaggacaaag |
| 601 | cccttcagag catggcgtcc atgtcctctg cccagatcgt ctctgccagt gtcctgcaga |
| 661 | acaagttcag cccaccttcc cctctgcccc aggccgtctt ctccacttcc tcgcggttct |
| 721 | ggagcagccc ccctctcctg ggacagcagc ctggaccctc tcaggacatc aagccctttg |
| 781 | cacagccagc ctaccccatc cagccgcccc tgccgccgac gctcagcagt tatgagcccc |
| 841 | tggccccgct cccctcagct gctgcctctg tgcctgtgtg gcaggaccgt accattgcct |
| 901 | cctcccggct gcggctcctg gagtattcag ccttcatgga ggtgcagcga gaccctgaca |
| 961 | cgtacagcaa acacctgttt gtgcacatcg gccagacgaa ccccgccttc tcagacccac |
| 1021 | ccctggaggc agtagatgtg cgccagatct atgacaaatt ccccgagaaa aagggaggat |
| 1081 | tgaaggagct ctatgagaag gggcccccta atgccttctt ccttgtcaag ttctgggccg |
| 1141 | acctcaacag caccatccag gagggcccgg gagccttcta tggggtcagc tctcagtaca |
| 1201 | gctctgctga tagcatgacc atcagcgtct ccaccaaggt gtgctccttt ggcaaacagg |
| 1261 | tggtagagaa ggtggagact gagtatgcca ggctggagaa cgggcgcttt gtgtaccgta |
| 1321 | tccaccgctc gcccatgtgc gagtacatga tcaacttcat ccacaagctg aagcacctgc |
| 1381 | ccgagaagta catgatgaac agcgtgctgg agaacttcac catcctgcag gtggtcacga |
| 1441 | gccgggactc ccaggagacc ctgcttgtca ttgcttttgt cttcgaagtc tccaccagtg |
| 1501 | agcacggggc ccagcaccat gtctacaagc tcgtcaaaga ctagggtgcc ctctgcgcct |
| 1561 | ccttaaggat gcagggtgag catctcctct ccacacctgc ctggcacccc tgggggggtc |
| 1621 | caggattgag gattcatcta cctgccaggc ctcaggccca ggacccagga ggcctcccca |
| 1681 | cctaccccag cacacacact ccctgccact gttctgcgct ttaattgtgg gagaagagag |
| 1741 | gagaggaggg ctcagcggtg gggcagcctg tccggggcgc tgacccacca tcaccctgct |
| 1801 | ctgcccagcc tcgcgtgacc tcagagaggt ggggataggg gacaccttca gcctccagca |
| 1861 | tgtgtggcca ctgtaccccc acccaccctt gggggagcat gatgggcagg tgagggcagg |
| 1921 | atggagacca agggagtcag tgagcagagg ccctgggagt gtccggttgg ggttggactg |
| 1981 | aggacagagg ggcccacact tccttgcccc tttgtgtccc aggcctggtg cccagactcc |
| 2041 | ttgcatggct tgtgtggtcc tcagactccg cacagcgagc gtaggtctct gggtttcaga |
| 2101 | tgaagtgccc aggctccagg aagttgaggg acccacagga gaggtgggca gagctggagt |
| 2161 | tctcatccag ggctgcttgt ccccagagcc caggtttata ctacctccct ggggcggggg |
| 2221 | ctggccgcag ggtaggggag aggctctgca gtgtggagtg gagcctcatc gaggggcgct |
| 2281 | gggttagggg agcacctgtt tcagactggg catgaagaag ggagcacagc agctactaga |
| 2341 | ccccattagc acctcattag cccacaagcc agccaggggc cccaggaaga tggggcaccc |
| 2401 | cccagcaccc tccagattga gagcaaggta gaggaaggag tcccagcctc tgggcagacc |
| 2461 | agaggcccag agggagagag tagcagaagg cttttgattt ttctcttgcc tgaggcttga |
| 2521 | atctgacaaa cccttggtgg gcactgctcc cttaggttct tccccacctc aatctacctg |
| 2581 | cctagagtag cagctcccag acccagttct gggactgaag gttaaccctt cacctgctgt |
| 2641 | cccttcttaa cacccaggcc cccagagcca gctgggcctg tccagcagcc acctgtgggt |
| 2701 | atttatgagt ttcatatgaa gtactgtgcc ccttcccttc ctcatcccga ccctgcccga |
| 2761 | gcttcctgaa ggtcctcact gtttgcatat cgctcaggcc acctccaaac cccacctagg |
| 2821 | ttttataatg tatattatat atttttttgt gtatttttaa aatccagctg tgatgggtta |
| 2881 | tatcataaat gcagcttggg gttggagcag gggccctcaa aggcccagct cctgctcaaa |
| 2941 | aaaaaaaaaa aaaaaaaatt aaagttattt gtttgtgggt cagtcatgta aaaaaaaaaa |
| 3001 | aaaaaaaaaa aaaaaaaaaa aaa |

By “TSC22D3 polypeptide” is meant a polypeptide or fragment thereof having at least about 85% or greater amino acid identity to the amino acid sequence provided at NCBI Accession No. NP_001305399.1 (isoform 1), NP_004080.2 (isoform 2), or NP_004080.2 (isoform 3) and having TSC22D3 polypeptide biological activity. Exemplary TSC22D3 biological activities include inhibition of NFAT/AP-1 transcription and inhibition of the mTORC2 pathway. An exemplary ID3 polypeptide sequence is provided below (NP_001305399.1) (SEQ ID NO:8):

| | |
|---|---|
| 1 | maqskldcrs pvgldccncc ldlahrsglq rgssgennnp gsptvsnfrq lgeklvfenl |
| 61 | ntdklnsimr qdslepvlrd pcylinegic nrnidqtmls illffhsasg asvvaidnki |
| 121 | eqamdlvknh lmyavreeve ilkeqirelv eknsqleren tllktlaspe qlekfqscls |
| 181 | peepapespq vpeapggsav |

By “TSC22D3 polynucleotide” is meant a polynucleotide encoding a TSC22D3 polypeptide. An exemplary ID3 polynucleotide sequence is provided at NCBI Accession No. NM_001318470.1 (SEQ ID NO: 9). SEQ ID NO: 9 is provided below:

| | |
|---|---|
| 1 | gtcatatccc agtgctgact cccgggcgtg cagaccgcta actagctcac tcgctctcag |
| 61 | ctcctgccac cgctcagccg tcacagccca ggggagcccg agagcctgag agcctgcaaa |
| 121 | ccggggaggg agggagcaaa ggagggaggg agcaagggcg cgccctggct ctccctctgc |
| 181 | cctgctgccc gcccttcctg cctccacagg caccctggag tcccctcagg ccagctcggt |
| 241 | gggcgcgcac ctgccagccg cccctgacct cgcaggccag gcgacctccg agcctgagaa |
| 301 | gatggcccag tccaagctcg attgccgctc acctgtcggc ctcgactgct gcaactgctg |
| 361 | cctggacctg gcccatcgga gtgggctcca gcgaggcagc agcggggaga acaacaaccc |
| 421 | gggcagccct acagtgagca actttcggca gctgcaggaa aagctggtct ttgagaacct |
| 481 | caataccgac aagctcaaca gcataatgcg gcaggattcg ctagagccgg tgctgcggga |
| 541 | cccctgctac ctgatcaacg agggcatctg caaccgcaac atcgaccaga ccatgctctc |
| 601 | catcctgctc ttcttccaca gtgcctccgg agccagcgtg gtggccatag acaacaagat |
| 661 | cgaacaggcc atggatctgg tgaagaatca tctgatgtat gctgtgagag aggaggtgga |
| 721 | gatcctgaag gagcagatcc gagagctggt ggagaagaac tcccagctag agcgtgagaa |
| 781 | caccctgttg aagaccctgg caagcccaga gcagctggag aagttccagt cctgtctgag |
| 841 | ccctgaagag ccagctcccg aatccccaca agtgcccgag gcccctggtg gttctgcggt |
| 901 | gtaagtggct ctgtcctcag ggtgggcaga gccactaaac ttgttttacc tagttctttc |
| 961 | cagtttgttt ttggctcccc aagcatcatc tcacgaggag aactttacac ctagcacagc |
| 1021 | tggtgccaag agatgtccta aggacatggc cacctgggtc cactccagcg acagacccct |
| 1081 | gacaagagca ggtctctgga ggctgagttg catggggcct agtaacacca agccagtgag |
| 1141 | cctctaatgc tactgcgccc tgggggctcc cagggcctgg gcaacttagc tgcaactggc |
| 1201 | aaaggagaag ggtagtttga ggtgtgacac cagtttgctc cagaaagttt aaggggtctg |
| 1261 | tttctcatct ccatggacat cttcaacagc ttcacctgac aacgactgtt cctatgaaga |
| 1321 | agccacttgt gttttaagca gaggcaacct ctctcttctc ctctgtttcg tgaaggcagg |
| 1381 | ggacacagat gggagagatt gagccaagtc agccttctgt tggttaatat ggtataatgc |
| 1441 | atggctttgt gcacagccca gtgtgggatt acagctttgg gatgaccgct tacaaagttc |
| 1501 | tgtttggtta gtattggcat agtttttcta tatagccata aatgcgtata tatacccata |
| 1561 | gggctagatc tgtatcttag tgtagcgatg tatacatata cacatccacc tacatgttga |
| 1621 | agggcctaac cagccttggg agtattgact ggtcccttac ctcttatggc taagtctttg |
| 1681 | actgtgttca tttaccaagt tgacccagtt tgtcttttag gttaagtaag actcgagagt |
| 1741 | aaaggcaagg aggggggcca gcctctgaat gcggccacgg atgccttgct gctgcaaccc |
| 1801 | tttccccagc tgtccactga aacgtgaagt cctgttttga atgccaaacc caccattcac |
| 1861 | tggtgctgac tacatagaat ggggttgaga gaagatcagt ttgggcttca cagtgtcatt |
| 1921 | tgaaaacgtt ttttgttttg ttttgtaatt attgtggaaa actttcaagt gaacagaagg |
| 1981 | atggtgtcct actgtggatg agggatgaac aaggggatgg ctttgatcca atggagcctg |
| 2041 | ggaggtgtgc ccagaaagct tgtctgtagc gggttttgtg agagtgaaca ctttccactt |
| 2101 | tttgacacct tatcctgatg tatggttcca ggatttggat tttgattttc caaatgtagc |
| 2161 | ttgaaatttc aataaacttt gctctgtttt tctaaaaata aaaaaaaaaa aaaaaaaaaa |
| 2221 | aaa |

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 is a plot showing response of cells as a function of puromycin concentration. HEK293T cells were transiently transfected with empty, wildtype, R183Q, or Q209L constructs and then exposed to puromycin for 3 days before cell number was assessed by MTT assay in 24 well plate. Half maximal inhibitory concentrations (IC₅₀) were 0.29 μM for empty and for wildtype, and 0.19 for R183Q and for Q209L. Two-way ANOVA demonstrated significant effects of both construct and dose (p<0.0001).

FIGS. 2A-2D are plots and images showing results of lentiviral infection with Empty, WT, or mutant constructs in HEK 293T and in EA.296 cells. FIG. 2A and FIG. 2B each shows that a mutant HEK293T cell line and a mutant EA.296 cell line respectively showed less cell survival (approximate percent survival) compared to empty or wildtype. FIG. 2C shows that expression of Gaq was less in mutant compared to Empty and WT in both cell lines; insufficient growth of the EA.296 Q209L was obtained to gather protein for western. FIG. 2D shows that cells with mutant construct showed more rounded morphology than wildtype or empty (images shown are from transient transfection in HEK 293T cells that showed this same morphology).

FIGS. 3A-3B are an image and plot showing overexpression of Gaq after transfection with GNAQ constructs. HEK 293T cells in six well plates were transiently transfected with pcDNA3.1-E, pcDNA3.1-GNAQ, pcDNA3.1-R183Q, pcDNA3.1-Q209L. 24 hours later, cells were digested and re-plated into 6-well plates. 24 hours later, the plates were incubated in DMEM (10% FBS). Three days later, protein was isolated. Gaq overexpression was demonstrated in cells transfected with the WT and mutant constructs. FIG. 3A shows results of a Western blot showing levels of Gaq in cells transfected with the WT and mutant constructs. FIG. 3B shows a quantification of the Western blot results shown in FIG. 3A.

FIGS. 4A-4B are an image and plot showing phosphorylated ERK in HEK 293T cells transiently transfected with GNAQ constructs. FIG. 4A shows results of a Western blot showing levels of p-ERK in cells transfected with the WT and mutant constructs. FIG. 4B shows a quantification of the Western blot results shown in FIG. 4A. P-ERK normalized to HSP90 (FIG. 4A) was significantly increased in mutant cells compared to WT (Panel B, *p<0.0).

FIG. 5 is a plot showing a dose response curve for puromycin. HEK 293T cells (5×10⁵ cells/well in six-well plate) were transiently transfected with pcDNA3.1-E, pcDNA3.1-GNAQ, pcDNA3.1-R183Q, pcDNA3.1-Q209L. Twenty-four (24) hours later, the cells were digested, counted and aliquoted into 96-well plates. Twenty-four (24) hours later, the cells were incubated with a series of puromycin concentrations. Three days later, the relative cell numbers were detected using a cell proliferation assay.

FIGS. 6A-6F are plots showing gene expression in WT and GNAQ mutant cells with and without puromycin. A panel of genes, important to the regulation of the pathways downstream of GNAQ, were evaluated by RT-PCR in mRNA samples gathered from cells with the pcDNA3.1-E, pcDNA3.1-GNAQ, pcDNA3.1-R183Q or pcDNA3.1-Q209L plasmids with or without puromycin treatment. FIG. 6A, FIG. 6B, FIG. 6C, FIG. 6D, FIG. 6E, and FIG. 6F each respectively show mRNA levels of TSC22D3, ID3, PIK3C2B, TEAD3, PRKCQ, and TRIO in cells transfected with WT and mutant constructs. Cells with mutant constructs were more sensitive to puromycin (0.4 μg/μl). TSC22D3 mRNA expression was decreased in cells with mutant constructs compared to WT and further decreased by puromycin exposure (FIG. 6A,*p<0.05). ID3 and TEAD3 expression were increased in mutants compared to WT and further increased by puromycin (FIG. 6B and FIG. 6D, *p<0.05).

FIG. 7 is a Western blot showing Gaq expression after puromycin treatment. Gaq protein expression was evaluated by RT-PCR in samples gathered from cells with the pcDNA3.1-E, pcDNA3.1-GNAQ, pcDNA3.1-R183Q or pcDNA3.1-Q209L plasmids with or without puromycin treatment. Cells with all GNAQ constructs treated with puromycin expressed similar levels of puromycin compared to untreated cells.

FIGS. 8A-8C are images and a plot showing p-ERK immunohistochemistry and p-ERK expression in SWS leptomeningeal vessel endothelial cells. FIG. 8A shows p-ERK immunohistochemistry and DAPI staining of blood vessel in the leptomeninges from subject with SWS. FIG. 8B shows p-ERK immunohistochemistry and DAPI staining of blood vessel in the leptomeninges from subject with Epilepsy (focal cortical dysplasia). FIG. 8C shows phosphorylated ERK expression was greater in endothelial cells from leptomeningeal vessels in subjects with SWS (*p<0.05).

### DETAILED DESCRIPTION OF THE INVENTION

The invention features compositions and methods that are useful for treating vascular malformations or related conditions. The invention is based, at least in part, on the discovery of increased puromycin sensitivity in cells transfected with a GNAQ containing a R183Q or Q209L mutation. R183Q mutations in GNAQ cause Sturge-Weber syndrome (SWS), a rare congenital neurocutaneous disorder, and capillary malformations (port-wine birthmarks), as well as uveal melanoma. Q209L or R183Q mutations in GNAQ cause uveal melanoma. Puromycin exposure was noted to impair efforts to establish stable cell lines with either the GNAQ R183Q or Q209L mutation. When puromycin was removed from the media, the efforts to establish stable cell lines with the mutations were successful. Based on these observations, experiments were performed to better understand the effect of these mutations and puromycin upon gene expression in cells transiently transfected with these GNAQ mutations.

HEK293T cells were transiently transfected with R183Q or Q209L constructs and a puromycin dose response curve completed. A dose of puromycin which partially inhibited cell growth was identified. Gene expression changes resulting from the mutations and from the puromycin treatment were assessed by RT-PCR and western analyses. Puromycin inhibited cell growth in cells with the R183Q and Q209L mutations. mRNA expression of ID3 and TEAD3 were up-regulated and TSC22D3 mRNA down-regulated significantly in R183Q or Q209L mutants compared to GNAQ wildtype. In addition, TSC22D3 expression was further down-regulated and ID3 expression was further up-regulated by puromycin treatment.

### Vascular Malformation/Sturge-Weber Syndrome

Capillary malformations/port-wine birthmarks occur in about 1 in 300 births and are very common. Sturge-Weber syndrome is a vascular malformation syndrome involving the brain, skin (capillary malformation/port-wine birthmark) and eye occurring in about 1 in 20,000 live births. Current treatments are primarily symptomatic and inadequate. The gene causing both Sturge-Weber syndrome and isolated port-wine birthmarks (capillary malformation of the skin not associated with brain or eye involvement) is the R183Q mutation in GNAQ. Recent evidence suggests that endothelial cells of the malformed blood vessels harbor the mutation (North et al., ISSVA 2014). Without being bound by theory, it is possible that other cell types also harbor the mutation.

### Capillary Malformations

Capillary malformations (port-wine stains) occur in about 1 in 300 live births and most of these occur on the head and neck regions. Some fading of the birthmark may be noted during the first year of life; however, these malformations are not self-resolving and require multiple courses of laser treatment with varying degrees of success. Capillary malformations frequently change from pink to red in early adulthood to a deep purple color later in adulthood. The surface may thicken (cobblestoning) and soft and bony hypertrophy may occur; these changes occur in about 60% of patients to varying degrees (Geronemus et al. (1991) J.Dermatol.Surg.Oncol.; 17 (1): 76-9). Nodular vascular lesions or pyogenic granulomas with bleeding can develop in adulthood. Psychosocial disability secondary to facial disfigurement can be severe, can worsen with adulthood, and include greater self-concern, self-doubt in interpersonal interactions, social inhibition, isolation, stigmatization from society, and limited opportunities compared to those without facial disfigurement (Geronemus et al. (1991) J.Dermatol.Surg.Oncol. 17 (1): 76-9). Less commonly acquired port-wine stains can occur at any age and are identical to congenital capillary malformations both clinically and histologically. Trauma, chronic UV exposure, and infections, have all been implicated in triggering the formation of acquired capillary malformations and a latent lesion brought out by trauma has been considered as a possible mechanism and acquired capillary malformation has been reported after trauma in the context of a congenital lesion (Tallman et al. (1991) Pediatrics 87 (3): 323-7).

Overall about 10% of facial capillary malformations are associated with vascular malformations in brain and/or eye (Sturge-Weber syndrome) but the risk primarily involves those infants born with a birthmark on the forehead, temple region, and/or upper eyelid (20-50%) (Comi (2011) Neurologist 17 (4): 179-84). Risk of glaucoma in these patients ranges from 25-75% (Tallman et al. (1991) Pediatrics 87 (3): 323-7) and the glaucoma can be refractory to available medical and surgical treatments resulting in vision loss. Brain involvement with the “leptomeningeal angioma” presents with seizures, strokes, migraines and focal neurologic impairments usually in the first two years of life (Shirley et al. (2013) N. Engl. J. Med. 368 (21): 1971-9), however about 10% of these individuals present later in childhood, adolescence or adulthood. Impaired venous drainage results in impaired brain perfusion which is exacerbated by seizures (Sujansky et al. (1995) J. Child Neurog. 10 (1): 49-58). The mainstay of current treatment for Sturge-weber syndrome is aggressive use of anticonvulsants. However medical management is effective in suppressing seizures in only about half of these patients, side effects are common, and neurologic impairments almost universal. Some patients have extensive, bilateral brain involvement and early onset of medically refractory seizures; for these patients especially the available treatments are very inadequate.

### Pathophysiology

The cause of both Sturge-Weber syndrome and isolated port-wine birthmarks is a R183Q somatic mosaic mutation in GNAQ in endothelial cells (Shirley et al. (2013) N. Engl. J. Med. 368 (21): 1971-9). Without being bound by theory, the mutation is predicted to impair autohydrolysis of the GTP binding site of Gaq thus maintaining the protein in an abnormally activated state. Transient transfection studies in HEK293T cells suggested that this mutation constitutively activates downstream pathways, with westerns demonstrating increased phosphorylated ERK and JNK (Shirley et al. (2013) N. Engl. J. Med. 368 (21): 1971-9). Histology demonstrates an increased number of capillary-venous vessels which dilate over time; the ectatic vessels tend to progress from the superficial dermis to the deeper dermis and subcutaneous tissues. Immunohistological studies of capillary malformations and Sturge-Weber syndrome brain tissue have implicated increased endothelial cell VEGF signaling. Vascular endothelial growth factor (VEGF)-A and its most active receptor VEGF-R2 expression are significantly increased in capillary malformation skin tissue compared with control skin (Comati et al. (2007) J. Neuropathol. Exp.Neurol. 66 (1): 86-97); similarly VEGF-R expression is increased in the endothelial cells of the malformed Sturge-Weber leptomeningeal vessels while mRNA expression levels of VEGF is increased in the underlying cortex (Comati et al. (2007) J. Neuropathol. Exp. Neurol. 66 (1): 86-97). Studies therefore suggest that venous stasis promotes surrounding tissue hypoxia and increases VEGF expression which may contribute to progression of the vascular malformation.

### GNAQ Mutations in Various Disorders

Hyperactivating mutations in GNAQ results in a growing number of disorders recently identified including uveal melanoma, blue nevi, Phakomatosis Pigmentovascularis and extensive dermal melanocytosis, melanocytic tumors originating in the central nervous system, low-grade glioma, port-wine birthmarks and Sturge-Weber syndrome (van de Nes et al. J.Neurooncol. 2016, doi: 10.1007/s11060-015-2052-2; Shirley et al., N. Engl. J. Med. 2013; 368:1971-1979; Van Raamsdonk et al., Nature 2009; 457:599-602; Thomas et al., J. Invest Dermatol. 2016, doi: 10.1016/j.jid.2015.11.027; Chan et al., Mod.Pathol. 2016, doi: 10.1038/modpathol.2015.153; Laviv et al., FEBS Open.Bio 2012; 2:129-134). GNAQ codes for Gaq, part of the trimeric G protein complex associated with a large subgroup of G protein coupled receptors (O'Hayre et al., Nat.Rev.Cancer 2013; 13:412-424). The R183Q and Q209L mutations are predicted to result in impaired auto-hydrolysis and therefore impaired deactivation of Gaq and constitutive hyperactivation of downstream pathways (Shirley et al., N. Engl. J. Med. 2013; 368:1971-1979; Van Raamsdonk et al., Nature 2009; 457:599-602). The involved hyperactivated pathways are beginning to be elucidated and include the Ras-Raf-MEK-ERK (Shirley et al., N. Engl. J. Med. 2013; 368:1971-1979), mTOR (Amirouchene-Angelozzi et al., Mol.Oncol. 2014; 8:1508-1520), and YAP-HIPPO pathways (Yu et al., Cancer Cell 2014; 25:822-830), however current understanding of the impact of these pathways upon gene expression is far from complete. Furthermore, efforts to identify novel targets or treatment approaches for capillary malformations, Sturge-Weber syndrome, uveal melanoma and other impacted tumors continue.

Sturge-Weber Syndrome (SWS), a sporadic neurocutaneous syndrome is classically associated with facial port-wine birthmark (PWB), with an ipsilateral vascular malformation in the eye causing glaucoma, and a leptomeningeal angioma involving the brain (Bachur et al., Curr.Treat.Options.Neurol. 2013; 15:607-617). The patients with Sturge-Weber Syndrome (SWS) present with clinical features including seizures, stroke-like episodes, and glaucoma because of vascular malformations involving the skin, brain, and eyes. Some patients display cognitive issues with attention issues/attention deficit hyperactivity disorder (Lance et al., Pediatr.Neurol. 2014; 51:675-680; Kavanaugh et al., Child Neuropsychol. 2015; 1-14. Isolated port-wine birthmarks occur in approximately 1:300 live births and consist of abnormal capillary-venous vessels in the dermis of the skin (Tallman et al., Pediatrics 1991; 87:323-327). The leptomeningeal vascular malformation consists of an increased number of tortuous vessels in the leptomeninges many of which are thin-wall and some of which are narrowed by sub-endothelial proliferation and hyalinization (Comati et al., J.Neuropathol.Exp.Neurol. 2007; 66:86-97; Di Trapani et al., Childs Brain 1982; 9:23-36). Recently both SWS and isolated port-wine birthmarks (capillary malformations) were shown to be associated with R183Q mutations in GNAQ (Shirley et al., N. Engl. J. Med. 2013; 368:1971-1979; Couto et al., Plast. Reconstr. Surg. 2016 January; 137 (1): 77e-82e. doi: 10.1097/PRS.0000000000001868; Nakashima et al., J.Hum.Genet. 2014; 59:691-693).

Both R183Q and Q209L GNAQ mutations have been demonstrated in uveal melanoma, with the Q209L mutation being the more common mutation (Van Raamsdonk et al., Nature 2009; 457:599-602). Prior studies have shown that the Q209L mutation is more activating than the R183 mutation (Shirley et al., N. Engl. J. Med. 2013; 368:1971-1979); the mutation is predicted to interfere more with the auto-hydrolysis site of the protein.

While pursuing efforts to establish stable endothelial cells (EA.hy926) lines with lentivirus plasmids and puromycin selection, it was noted that both R183Q and Q209L infected cells barely survived puromycin selection, whereas most of those cells infected by empty and wildtype GNAQ plasmids survived under the same conditions. Work with human microvascular endothelial cells (HMEC) to produce stable cell lines with puromycin selection also resulted in fewer clones, with less robust protein expression and impaired growth when the cells were maintained with puromycin. These observations suggested that the R183Q and Q209L mutations may induce enhanced cell vulnerability to puromycin. Therefore transient transfection of pcDNA3.1-E,-GNAQ,-R183Q and Q209L into HEK293T cells was performed, and then a puromycin dose response curve was performed, followed by RT-PCR and western analyses for gene expression changes resulting from the mutant transfections combined with puromycin exposure. Results of these experiments are described elsewhere herein.

### GNAQ and Cellular Signaling

An interaction, between the effects of hyperactivating GNAQ mutations and cellular insults from exposure to puromycin, has been previously described; however this interaction has been previously studied from a very different perspective and in a very different context. Wang et. al. studied the puromycin aminonucleoside nephrosis (PAN model) of focal segmental glomerulosclerosis both in vitro and in vivo, and combined this with expression of the constitutively hyperactive Q209L GNAQ mutation. They found that the Q209L mutation alone was insufficient to produce injury, however adding exposure to puromycin resulted in cellular injury, albuminurea, and focal segmental glomerulosclerosis which was more severe than in the Q209L animals treated with vehicle. Using a calcineurin/NFAT and Q209L reporter mouse they showed that GNAQ hyperactivation signaled through NFAT. An important gene target of NFAT signaling is Transient Receptor Potential Channel 6 (TRPC6) activity, an ion channel with activity that was increased by Gaq induction and inhibited by the calcineurin inhibitor FK506. TRPC6 knockout Q209L mice treated with puromycin did not demonstrate the increased susceptibility to puromycin suggesting that the enhance puromycin sensitivity was at least partially reliant on TRPC6 activity (Wang, J Clinical Invest 2015 May;125 (5): 1913-26).

TRP channels are cation-permeable channels broadly expressed in organisms and tissue types, including the brain and the vasculature. TRP channels mediate a wide range of physiological functions including cell cycle regulation, cell apoptosis and survival. Endothelial cells express several transient receptor potential isoforms; their activity modulates cytosolic calcium levels and membrane potential (Kwan et al. Biochemica et Biophysica Acta 2007 August; 1772 (8): 907-14). Gaq activation of TRPC6 signals the activation of PKCa which then induces RhoA activity, endothelial cell contraction (Singh et al 2007, J Biol Chem March 16;282 (11): 7833-43), and resulting in endothelial barrier dysfunction. Interestingly, a rounded shape with inter-endothelial gaps is described in the abnormal endothelial cells of both port-wine blood vessels and of the leptomeningeal vessels in Sturge-Weber syndrome and noted in this study as well. Contrast enhancement on MRI imaging is used to diagnose the leptomeningeal angioma in SWS; this clinical finding also suggests endothelial barrier dysfunction. Inhibiting excessive TRPC6 signaling may result in improved endothelial barrier and vascular function. Furthermore, TRPC6 is associated with pressure related diseases in many conditions and its expression can be induced by mechanical stimulation. The intravascular pressure-induced depolarization and constriction of small arteries and arterioles are regulated by TRPC6 and its expression is increased in pulmonary hypertension (Lin et al., Circ Res. 2004; 95 (5): 496-505).

TRPC6 complexes with other proteins and appears to form an environmental pressure sensor. This has generated interest regarding the role of TRPC6 in glaucoma (Fan et al., Int J Ophthalmol. 2012; 5 (4): 523-526) and suggests a role for this protein in the response to capillary-venous engorgement and impaired function in capillary malformations and Sturge-Weber syndrome. Endothelial TRPC6 contributes to VEGF-induced calcium influx in microvessel endothelial cells (Hamdollah Zadeh et al., Microcirculation. 2008 October; 15 (7): 605-614). Therefore, excessive activation could result in impaired cellular function, and inhibiting this pathway theoretically could be protective.

Ca++influx via TRPC6 also activates calcineurin, increases ERK phosphorylation and increases NFAT expression. NFAT is known to induce the activity of inhibitor of DNA binding (ID3), at least in some contexts. Koltsova et al. reported Egr1 and NFAT act together to promote the development of T-cells and cooperatively induce the expression of ID3 (Koltsova et al., Biochemistry (Mosc). 2007 September;72 (9): 954-61). The ERK and Ca++signaling pathways act in concert by converging on the NFAT pathway. Ids are a small family of helix-loop-helix proteins that lack the ability to interact with DNA but act as dominant-negative transcription factors, and regulate a variety of cellular functions including cell cycle progression, proliferation, migration, angiogenesis, and invasion. Upregulation has been found in a variety of cancers, including melanoma (DiVito et al 2014, Carcinogenesis, Apr;35 (4): 951-8).

ID3 is pro-angiogenic and it has been suggested as a therapeutic target for the treatment of melanoma and several other cancers where ID3 expression is increased (DiVito et al 2014, Carcinogenesis, Apr;35 (4): 951-8). ID1 and ID3 expression regulated by Akl1 are both necessary for full induction of EphrinB2, itself critical to driving blood vessels to either venular phenotype (EphrinB2-) or to arteriolar phenotype (EphrinB2+) (Kim et al. 2012 Angiogenesis. 2012 September; 15 (3): 497-509). In T-helper cells, ID3 modulates the activities of the PI3K-AKT-mTORC1-HIF 1 a pathway to modulate cellular proliferation. In endothelial cells, Id1 and Id3 are induced by VEGF and TGFbeta and overexpression of these Id proteins enhance MMP2 and MMp9 expression and tube formation (Sakurai et al 2004, J Immunol. 2004 Nov. 1;173 (9): 5801-9.). ID protein inhibition has been suggested as a target for treatment in vascular malformations. The results in this study suggest that overexpression of mutant Gaq results in increased ID3 expression and we hypothesize that this may have a role in the abnormal vascular structure and function of the leptomeningeal blood vessels. Constitutively increased ID3 expression furthermore may contribute to increased sensitivity to puromycin toxicity. Further studies are needed to address this hypothesis.

TSC22D3 (also known as GILZ) is a glucocorticoid induced leucine zipper gene. TSC22D3 inhibits NFAT/AP-1 transcription (de Bosscher et al. 2003, Endocr Rev. 2003 August;24 (4): 488-522), interacts directly with c-Fos and c-Jun, and inhibits Raf-1 phosphorylation (and thereby suppress MEK and ERK phosphorylation) in normal T-cells (Ayroldi et al 2002 Mol Cell Biol. Nov;22 (22): 7929-41). TSC22D3 expression is induced by glucocorticoid (corticosteroid) treatment in airway epithelial cells (Eddleston et al. 2007, J Allergy Clin Immunol. Jan; 119 (1): 115-22) and smooth muscle cells (Kelly et al. 2012 Br J Pharmacol. Mar; 165 (6): 1737-47). GILZ is a key inhibitor of the mTORC2 pathway and reduces AKT (Joha 2012 Oncogene. March 15;31 (11): 1419-30). GILZ over-expression in microvascular endothelial cells inhibited TNF-α induced activation of p38, ERk, and JNK MAPKs (Cheng et al. 2013 J Immunol. July 1;191 (1): 424-33). Here TSC22D3 expression was decreased in the GNAQ mutants and further decreased by puromycin in all the cells. Without being bound by theory, decreased TSC22D3 expression is believed to contribute to hyperactivation of the MEK-ERK pathway and therefore enhance susceptibility to puromycin toxicity.

Corticosteroids are used intermittently in patients with Sturge-Weber syndrome when anticonvulsants and other acute management fail to bring prolonged episodes of seizures, migraines and stroke-like episodes under control. One function of corticosteroids in the treatment of Sturge-Weber syndrome may be to increase the inhibition of these pathways through increased expression of GILZ. Chronic steroid use has significant medical complications and therefore identification of drugs lacking off target effects is currently underway for a number of conditions impacted by these pathways that should include uveal melanoma, Sturge-Weber syndrome and port-wine birthmarks.

Transcriptional enhancer factor (TEF-5) is the protein that in humans is encoded by the TEAD3 gene. It is a member of the transcriptional enhancer factor (TEF) family of transcription factors which contain the TEA/ATTS DNA-binding domain. It plays a key role in the Hippo signaling pathway, a pathway involved in organ size control and tumor suppression by restricting proliferation and promoting apoptosis. The pathway is essentially composed of a kinase cascade where MST1/MST2, in complex with its regulatory protein SAVI, phosphorylates and activates LATS1/2 complexed with its regulatory protein MOB1, which in turn phosphorylates and inactivates the YAPI oncoprotein and WWTR1/TAZ. TEF-5 acts by mediating gene expression of YAPI and WWTR1/TAZ to regulate cell proliferation and migration. It binds to multiple functional elements of the human chorionic somatomammotropin-B gene enhancer and normally it is predominately expressed in the placenta, but is also expressed in the nervous system and muscles of embryonic fish, and in the early developing mouse heart. TEF-5 is important in the transactivation of the chorionic somatomammotropin-B gene enhancer (also called human placental lactogen; hPL) which has weak actions similar to growth hormone although 100 times greater amounts are required to produce the same effect. a1-adrenergic receptor activity in neonatal mouse cardiac myocytes increases TEF-5 activity, suggesting a role in the signaling downstream of Gaq.

### GNAQ and Clinical Relevance

Previous studies of molecular neuropathology in Sturge-Weber syndrome provide an important context for the interpretation of these results. Comati et al. reported in 2007 that the majority of the vessels were thin walled vessels of variable caliber, ectatic, CD34+, and covered by a layer of smooth muscle/pericytes (Comati et al., J Neuropathol Exp Neurol. 2007 January;66 (1): 86-97). Most SWS vessels did not have an internal elastic lamina, as indicated by Elastica van Gieson stain indicating that the leptomeningeal angioma primarily consists of vessels with venous characteristics. Arterial vessels with an internal elastic lamina were scattered within the leptoangiomatous lesion. Compared to control leptomeningeal vessels, and cortical vessels from the same SWS samples, these SWS leptomeningeal vessels expressed greater amounts of VEGFR-1 and VEGFR-1 and HIF1α and HIF2α. They suggested a model whereby increased VEGF released by the hypoxic cortex stimulated the increased release of HIF1α and further increases in VEGF. A greater mitotic index for the endothelial cells of these vessels was also noted suggesting ongoing vascular remodeling. Decreased protein levels of fibronectin expression in SWS leptomeningeal vessels have also been reported. VEGF and VEGFR signal through Gaq, and therefore the hyperactivating R183Q mutation in GNAQ may increase the expression of HIF1α and this data suggests that increased ID3 expression may be involved.

In port-wine birthmarks, increased endothelial cell p-ERK expression has also been reported and suggested to contribute to early morphological vascular structural and functional abnormalities (Tan et al 2014 J Am Acad Dermatol. Nov;71 (5): 964-8). Adult and hypertrophied port-wine birthmarks are reported to also demonstrate increase expression of other downstream kinases suggesting that progressive hyperactivation of these pathways may contribute to the vascular ectasia and birthmark hypertrophy that can occur over time. It has also been suggested that increased expression of VEGF and VEGF expression contributes to vascular hypertrophy in port-wine birthmarks (Vural et al Otolaryngol Head Neck Surg. 2008 October; 139 (4): 560-4). Puromycin is an aminoglycocide antibiotic that is utilized frequently in the lab for selection of transfected cells with a gene conveying antibiotic resistance. Its toxicity in non-resistant cells is generally understood to result from its inhibition of protein transcription. However it also inhibits Puromycin-sensitive aminopeptidase (PSA; also called NPEPPS) which contains the zinc-binding domain characteristic of the gluzincin group of zinc metalloproteases. NPEPPS is an aminopeptidase with broad substrate specificity for several peptides. It is involved in proteolytic events essential for cell growth and viability. It may act as regulator of neuropeptide activity, have a role in the antigen-processing pathway for MHC class I molecules and be involved in the N-terminal trimming of cytotoxic T-cell epitope precursors. It digests the poly-Q peptides found in many cellular proteins.

Constitutively increased ID3 expression in the GNAQ mutants in the studies herein was associated with increased sensitivity to puromycin toxicity; without being bound by theory, constitutively increased ID3 may increase HIF1α expression and drive further VEGF release. Similarly, in the studies herein, decreased TSC22D3 expression was associated with increased puromycin susceptibility.

Puromycin's toxicity limits its clinical usefulness, although it has, in the past, been studied in a few clinical cancer trials. Here the value of puromycin is primarily as a metabolic stressor highlighting that the GNAQ mutations increase cell vulnerability. Considering treatment strategies, without being bound by theory, puromycin (or a safer analogue) administered topically after laser treatment of a capillary malformation (port-wine birthmark) would preferentially reduce regrowth of abnormal blood vessels. It is also a possible treatment strategy for uveal melanoma.

Bestatin (ubenimex, Eiger BioPharmaceuticals, Inc.), is an oral, competitive, reversible protease inhibitor of the leukotriene A+hydrolase (LTA+H), an enzyme that converts LTA+ to LTB+, and an inflammatory mediator that occurs naturally. It has also been shown to inhibit PSA and to inhibit cell proliferation. Bestatin has been marketed in Japan for more than 25 years for the treatment of Pulmonary Arterial Hypertension (PAH) and other inflammatory diseases. It is currently in clinical trials for the treatment of AML. If PSA inhibition is important to the increase puromycin sensitivity of the GNAQ mutants observed here, the bestatin, a drug current in clinical trials may mimic these effects.

Ultimately the goal of translational research is to identify novel molecular targets and treatment strategies for clinical conditions. Without being bound by theory, data herein indicate that puromycin analogues, or other drugs targeting ID3, TSC22D3 or PSA (such as Bestatin) could provide novel targets for further study. These studies have been crucial to the success of efforts to establish stable endothelial cell lines with these GNAQ mutations. With these stable cell lines, efforts have now begun to further study these targets and carry out dose response curves to test the ability of drugs to induce cell death or normalize cellular function.

### Puromycin and Cellular Signaling

Puromycin is an aminonucleoside known to have toxic effects upon cells. It is an antibiotic commonly used in stable cell line selection. Recently reported work in mice with GqQ>L (Q209L) induction in podocytes exposed to puromycin (model for a kidney disease called focal glomerular sclerosis) developed more glomerular injury compared to control animals and linked this finding to TRPC6 signaling (Wang et al. (2015) J. Clin. Invest 125 (5): 1913-26). TRPC6 channels are Ca2+ permeable non-selective cation channels that can be activated by both G-protein coupled receptors (GPCR) and by receptor tyrosine kinases via phospholipase C (PLC) and diacylglycerol (DAG) mediated signaling. In podocytes with the induced Q209L mutation TRPC6 activation did not cause glomerular damage in the absence of an additional cell stressor.

### Increased Sensitivity to Puromycin in GNAQ R183Q Cells

The present invention features a method of inhibiting proliferation and/or reducing survival of a cell comprising a GNAQ polynucleotide or polypeptide having a R183Q or Q209L mutation, comprising contacting the cell with puromycin or a puromycin analog. In the process of establishing HEK293 and endothelial cell lines with the R183Q and Q209L GNAQ mutations, an unexpected finding which has several important translational implications was stumbled upon. Wildtype, R183Q, and Q209L GNAQ constructs were inserted into a lentivirus-plasmid, the sequences were confirmed and after transfection, and puromycin selection was done to generate stable HEK293 and EA.hy 296 (endothelial) cell lines. In the HEK293 cells, 90-100% of the cells with empty or wildtype GNAQ construct survived the selection whereas only 75% of the cells with the R183Q and 40% of the Q209L mutations survived the selection. In the EA.hy 926 endothelial cells 100% of the cells with the empty or wildtype constructs, but only 30% of those with the R183Q and less than <5% of those with the Q209L mutation survived the puromycin selection. This amount of cell death after puromycin selection was unexpected. Without wishing to be bound by theory, it was possible that the cells did not tolerate the transfection with multiple copies of the ONAQ mutations. However, this much cell death after transient transfection into these cells with the GNAQ mutant constructs was not previously observed. Without being bound by theory, an alternative explanation was that puromycin was more toxic to cells with the mutations.

The studies described herein are the first to link puromycin sensitivity to the R183Q and Q209L GNAQ mutation in endothelial cells and vascular malformations and related syndromes, and to suggest use of this antibiotic, its analogs, or its inhibitors for the treatment of capillary malformations and related syndromes. Furthermore, the finding indicates that human endothelial cells with the R183Q GNAQ mutation are puromycin sensitive, implying that capillary malformations may be treated with puromycin (or puromycin analog) applied topically, either following laser treatment or as the sole treatment to cause fading of the birthmark or to prevent the progression (blebbing, soft tissue hypertrophy). Accordingly, the present invention provides methods of reducing a vascular malformation in a subject, inhibiting progression of a vascular malformation in a subject, reducing appearance of a birthmark in a subject, and treating a vascular malformation or related condition in a subject. The methods comprise administering to the subject an effective amount of puromycin or a puromycin analog.

This finding also has immediate implications for efforts to generate cell culture models of capillary malformations/Sturge-Weber syndrome since it is common to maintain stable cell lines in media with puromycin after selection. Thus, the present invention features cell lines comprising an isolated polynucleotide encoding a GNAQ polypeptide comprising a R183Q or Q209L mutation. In some embodiments, the cell lines further comprise an isolated polynucleotide encoding a puromycin resistance polypeptide.

Finally, puromycin sensitivity may also serve as a suitable and titratable in vitro assay for identification of drugs which block this effect. Candidate drugs blocking or enhancing puromycin sensitivity could prove useful for treatment of vascular malformations. Accordingly, the present invention features methods of identifying candidate agents that modulate a vascular malformation. The screening methods comprise contacting a cell comprising a GNAQ polynucleotide or polypeptide having a R183Q or Q209L mutation with puromycin and a candidate agent; and comparing viability of the contacted cell with a reference level of viability. The screening methods may also comprise comparing levels of GNAQ polypeptide or polynucleotide and levels of polypeptides or polynucleotides of genes downstream of GNAQ polypeptide (or Gaq) dependent signaling pathways.

Treatment of Vascular Malformation and Related Conditions with Puromycin

Puromycin was identified as an agent useful for preventing or ameliorating a disease associated with a GNAQ R183Q mutation. Diseases associated with a GNAQ R183Q mutation include, without limitation, vascular malformation, vascular malformation in the eye, vascular malformation in the brain, capillary malformation, Sturge-Weber syndrome, and uveal melanoma. For example, the cause of both Sturge-Weber syndrome and isolated port-wine birthmarks is a R183Q somatic mosaic mutation in GNAQ in endothelial cells. The same mutation in melanoma cells causes uveal melanoma.

Accordingly, the present invention provides methods of treating disease associated with a GNAQ Q209L or R183Q mutation and/or disorders or symptoms thereof which comprise administering a therapeutically effective amount of a pharmaceutical composition comprising a puromycin or puromycin analog to a subject (e.g., a mammal such as a human). One embodiment is a method of treating a subject suffering from or susceptible to a disease associated with GNAQ R183Q mutation (e.g., vascular malformation, vascular malformation in the eye, vascular malformation in the brain, capillary malformation, Sturge-Weber syndrome, and uveal melanoma) or disorder or symptom thereof. The method includes the step of administering to the mammal a therapeutic amount of puromycin or a puromycin analog sufficient to treat the disease or disorder or symptom thereof, under conditions such that the disease or disorder is treated.

The methods herein include administering to the subject (including a subject identified as in need of such treatment) an effective amount of a puromycin or puromycin analog, or a composition described herein to produce such effect. Identifying a subject in need of such treatment can be in the judgment of a subject or a health care professional and can be subjective (e.g. opinion) or objective (e.g. measurable by a test or diagnostic method).

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

The therapeutic methods of the invention (which include prophylactic treatment) in general comprise administration of a therapeutically effective amount of puromycin or puromycin analog to a subject (e.g., animal, human) in need thereof, including a mammal, particularly a human. Such treatment will be suitably administered to subjects, particularly humans, suffering from, having, susceptible to, or at risk for vascular malformation, vascular malformation in the eye, vascular malformation in the brain, capillary malformation, Sturge-Weber syndrome, and uveal melanoma. In particular embodiments, the puromycin or puromycin analog is administered to a subject having a capillary malformation (“port wine stain” or “port wine birthmark”) to cause fading of the birthmark. In particular embodiments, the puromycin or puromycin analog is administered to a subject having a vascular malformation to prevent the progression of the disease (e.g., blebbing, soft tissue hypertrophy).

Determination of those subjects “at risk” can be made by any objective or subjective determination by a diagnostic test or opinion of a subject or health care provider (e.g., genetic test (particularly, genetic test for GNAQ R183Q mutation), enzyme or protein marker, family history, and the like). The puromycin compositions herein may be also used in the treatment of any other disorders in which the GNAQ R183Q mutation may be implicated.

In some embodiments, a subject is selected for treatment with puromycin or a puromycin analog by detection of a GNAQ R183Q mutation in a sample obtained from the subject. The sample obtained from the subject may be a sample of endothelial cells from a capillary malformation in the subject. Methods for detecting a GNAQ R183Q mutation in the sample include immunoassay, direct sequencing, and probe hybridization to a polynucleotide encoding the mutant polypeptide.

The administration of a therapeutic composition comprising puromycin or a puromycin analog may be by any suitable means that results in a concentration of the therapeutic that, combined with other components, is effective in ameliorating, reducing, or stabilizing a GNAQ R183Q mutation-associated disease (e.g., vascular malformation, vascular malformation in the eye, vascular malformation in the brain, capillary malformation, Sturge-Weber syndrome, and uveal melanoma). The therapeutic composition comprising puromycin or a puromycin analog may be administered systemically, for example, formulated in a pharmaceutically-acceptable buffer such as physiological saline. The therapeutic composition comprising puromycin or a puromycin analog may also be administered topically. Routes of administration include, for example, subcutaneous, intravenous, interperitoneally, intramuscular, or intradermal injections that provide continuous, sustained levels of the drug in the patient.

Treatment of human patients or other animals is carried out using a therapeutically effective amount of puromycin or a puromycin analog in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the GNAQ R183Q mutation-associated disease. Generally, amounts will be in the range of those used for other agents used in the treatment of the GNAQ R183Q mutation-associated disease, although in certain instances lower amounts will be needed because of the increased specificity of the compound. The therapeutic composition is administered at a dosage that ameliorates the GNAQ R183Q mutation-associated disease and/or symptoms thereof (e.g., reduces the vascular malformation or reduces appearance of a birthmark) as determined by a method known to one skilled in the art.

The therapeutic agent may be contained in any appropriate amount in any suitable carrier substance, and is generally present in an amount of 1-95% by weight of the total weight of the composition. The composition may be provided in a dosage form that is suitable for parenteral (e.g., parenterally by injection) administration route. The composition may be provided in a dosage form that is suitable for topical administration or ocular administration. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Compositions may be provided in unit dosage forms (e.g., in single-dose ampoules), or in vials containing several doses and in which a suitable preservative may be added. The composition may be in the form of a solution (e.g., eye drops, spray, oil), a suspension (e.g., gel, hydrogel, ointment, paste), an emulsion, a dermal application (e.g., topical cream, liniments, film, patch, lotion, balm), or any appropriate method known in the art. It may be presented as a dry powder (e.g., effervescent powder) to be reconstituted with water or another suitable vehicle before use. Apart from the active agent(s) that reduces or ameliorates a GNAQ R183Q mutation-associated disease (e.g., a vascular malformation or related condition), the composition may include suitable parenterally acceptable carriers and/or excipients. Furthermore, the composition may include suspending, solubilizing, stabilizing, pH-adjusting agents, tonicity adjusting agents, and/or dispersing, agents.

In particular embodiments, the composition is formulated for ocular or ophthalmic administration. In particular embodiments, the composition is in the form of a solution, particularly a solution suitable for ophthalmic application (e.g., eye drops). In particular embodiments, the composition is formulated for topical administration. In particular embodiments, the composition is in the form for dermal application (e.g., a topical cream). In compositions suitable for dermal application, the puromycin or puromycin analog may be incorporated with petroleum jelly, beeswax, paraffin, polyethylene glycol, gelatin, or the like.

In particular embodiments, the composition is formulated for administration by injection. Pharmaceutical compositions according to the invention may be prepared in the form suitable for sterile injection. To prepare such a composition, the puromycin or puromycin analog is dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

In particular embodiments, the composition is formulated for oral administration. Formulations for oral administration include tablets containing puromycin or a puromycin analog in a mixture with non-toxic pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active drug in a predetermined pattern (e.g., in order to achieve a controlled release formulation) or it may be adapted not to release the active drug until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material, such as, e.g., glyceryl monostearate or glyceryl distearate may be employed. The solid tablet compositions may include a coating adapted to protect the composition from unwanted chemical changes, (e.g., chemical degradation prior to the release of the active therapeutic substance). The coating may be applied on the solid dosage form in a similar manner as that described in Encyclopedia of Pharmaceutical Technology, supra.

### Combination Therapies

In some embodiments, the therapeutic composition comprising puromycin or a puromycin analog may be administered to a subject having vascular malformation or related condition, in combination with any other standard therapy for the disease. Standard therapy for vascular malformation includes, for example, laser treatment.

### Kits

The invention provides kits for the treatment or prevention of a GNAQ R183Q mutation-associated disease (e.g., vascular malformation, vascular malformation in the eye, vascular malformation in the brain, capillary malformation, Sturge-Weber syndrome, and uveal melanoma). In one embodiment, the kit includes a therapeutic or prophylactic composition containing an effective amount of puromycin or puromycin analog. In some embodiments, the kit comprises a sterile container which contains the therapeutic or prophylactic composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired, the therapeutic or prophylactic composition is provided together with instructions for administering the puromycin or puromycin analog to a subject having or at risk of developing a GNAQ R183Q mutation-associated disease (e.g., vascular malformation, vascular malformation in the eye, vascular malformation in the brain, capillary malformation, Sturge-Weber syndrome, and uveal melanoma). The instructions will generally include information about the use of the composition for the treatment or prevention of the GNAQ R183Q mutation-associated disease. In other embodiments, the instructions include at least one of the following: description of puromycin or puromycin analog; dosage schedule and administration for treatment or prevention of vascular malformation or related conditions or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

### Cell Lines Expressing Mutant GNAQ

The present invention provides recombinant human embryonic kidney (HEK) or endothelial cell lines comprising an isolated polynucleotide encoding a GNAQ polypeptide comprising a R183Q mutation. Such cell lines may be useful for screening candidate agents that modulate vascular malformation or a related condition.

Those skilled in the field of molecular biology will understand that any of a wide variety of expression systems may be used to provide a cell line heterologously expressing GNAQ polypeptide comprising a R183Q mutation. The precise host cell used is not critical to the invention. A polypeptide of the invention may be produced in mammalian cells (e.g., HEK cells, endothelial cells). Such cells are available from a wide range of sources (e.g., the American Type Culture Collection, Rockland, Md.; also, see, e.g., Ausubel et al., Current Protocol in Molecular Biology, New York: John Wiley and Sons, 1997). The method of transformation or transfection and the choice of expression vehicle will depend on the host system selected. Transformation and transfection methods are described, e.g., in Ausubel et al. (supra); expression vehicles may be chosen from those provided, e.g., in Cloning Vectors: A Laboratory Manual (P. H. Pouwels et al., 1985, Supp. 1987). In particular embodiments, the cells are transiently transfected with expression vectors for producing mutant GNAQ polypeptides. In particular embodiments, the cells are stably transfected with expression vectors for producing mutant GNAQ polypeptides. In particular embodiments, the cells are HEK293, HEK293T, EA.926, EA.hy 926, or HUVEC.

A variety of expression systems exist for heterologous expression of mutant GNAQ polypeptides. Expression vectors useful for producing such polypeptides include, without limitation, virus-derived vectors, e.g., vectors derived from viruses such as baculoviruses, papova viruses, such as SV40, vaccinia viruses, adenoviruses, fowl pox viruses, pseudorabies viruses and retroviruses, and vectors derived from combinations thereof. In particular embodiments, the vector is a lentivirus plasmid.

### Screening Assays

Methods of the invention are useful for the high-throughput low-cost screening of candidate agents that modulate a GNAQ R183Q mutation-associated disease, such as vascular malformation. Screening assays of the invention are based, at least in part, on the discovery of a link between puromycin sensitivity and the GNAQ R183Q mutation. Cells with hyperactivating GNAQ mutations have increased vulnerability to puromycin; recognizing this is essential to efforts to establish and maintain stable cell lines with these mutations. The mutations altered expression of proteins impacting molecular pathways downstream of Gaq and critical to angiogenesis, cell differentiation and cell survival. Puromycin further altered expression of these proteins impacting proteins which offer insights into possible novel targets for drug development.

Without intending to be bound by theory, it is believed that puromycin sensitivity in GNAQ R183Q mutant cells is linked to the same cellular signaling pathway(s) implicated in vascular malformations and other GNAQ R183Q mutation-associated diseases. Thus, candidate drugs identified could prove useful for treatment of vascular malformations. In particular embodiments, the screening assay comprises (a) contacting a cell comprising a GNAQ polynucleotide or polypeptide having a R183Q mutation with puromycin and a candidate agent; and (b) comparing viability of the contacted cell with a reference level of viability. In particular embodiment, an alteration in viability indicates that the candidate agent modulates a GNAQ R183Q mutation-associated disease. In particular embodiments, an alteration in viability indicates that the candidate agent modulates a vascular malformation.

Results of studies herein also indicate that ID3, TSC22D3, and TEAD3 are linked to the same cellular signaling pathway(s) implicated in vascular malformations and other GNAQ R183Q mutation-associated diseases. Accordingly, the in another aspect, the invention provides a method of identifying an agent that modulates a vascular malformation or related condition, where the method contains the steps of (a) contacting a cell with a candidate agent, and (b) measuring a level or activity of a ID3, TSC22D3, or TEAD3 polynucleotide or polypeptide, where an alteration in the level or activity of the ID3, TSC22D3, or TEAD3 polynucleotide or polypeptide indicates that the candidate agent modulates a vascular malformation or related condition.

Cell lines according to the invention (e.g., HEK or endothelial cells comprising an isolated polynucleotide encoding a GNAQ polypeptide comprising a R183Q mutation) may be used in the screening assays. The viability of a cell contacted with a candidate agent may be measured using cell viability assays known in the art. Assays for measuring cell viability are known in the art, and are described, for example, by Crouch et al. (J. Immunol, Meth. 160, 81-8): Kangas et al. (Med. Biol.62, 338-43, 1984); Lundin et al., (Meth. Enzymol.133, 27-42, 1986); Petty et al. (Comparison of J. Biolum. Chemilum. 10, 29-34,.1995); and Cree et al. (AntiCancer Drugs 6:398-404, 1995). Cell viability can be assayed using a variety of methods, including MTT (3-(4,5-dimethylthiazolyl)-2,5-diphenyltetrazolium bromide) (Barltrop, Bioorg. & Med. Chem. Lett. 1:611, 1991; Cory et al., Cancer Comm. 3, 207-12, 1991; Paull J.

Heterocyclic Chem. 25, 911, 1988). Assays for cell viability are also available commercially. These assays include but are not limited to CELLTITER-GLO® Luminescent Cell Viability Assay (Promega), which uses luciferase technology to detect ATP and quantify the health or number of cells in culture, and the CellTiter-Glo® Luminescent Cell Viability Assay, which is a lactate dehyrodgenase (LDH) cytotoxicity assay (Promega). In particular embodiments, the cell viability is measured using an MTT assay.

One skilled in the art appreciates that the effects of a candidate agent on a cell is typically compared to a corresponding control cell not contacted with the candidate agent. Thus, the screening methods include comparing the proliferation of a cell comprising a GNAQ R183Q mutation contacted by a candidate agent to the proliferation of an untreated control cell. The viability of cells contacted with puromycin and a candidate agent may be compared with a reference level of viability. For example, a reference level of viability may be the viability of cells not contacted with the candidate agent and contacted with puromycin only. In particular embodiments, the alteration in viability is positive (i.e., cells contacted with the candidate agent and puromycin have increased viability compared to cells contacted with puromycin only). In particular embodiments, the alteration in viability is negative (i.e., cells contacted with the candidate agent and puromycin have decreased viability compared to cells contacted with puromycin only).

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Cells Harboring GNAQ R183Q or Q209L Mutation had Increased Cell Death After Transfection with GNAQ R183Q or Q209L Construct Using Puromycin Selection

In the process of establishing HEK and endothelial cell lines with the R183Q and Q209L GNAQ mutations, an unexpected finding which potentially has several important translational implications was stumbled upon. Wildtype, R183Q, and Q209L GNAQ mutants were inserted into a lentivirus-plasmid. The sequences were confirmed and after transfection, puromycin selection done to generate stable HEK (human embryonic kidney) and EA.296 (endothelial) cell lines. In the HEK293 cells, 90-100% of the cells with empty or WT GNAQ survived the selection whereas only 75% of the cells with the R183Q and 40% of the Q209L mutations survived the selection. In the EA.926 endothelial cells 100% of the cells with the empty or WT constructs, but only 30% of those with the R183Q and less than <5% of those with the Q209L survived the puromycin selection.

The amount of cell death after puromycin selection was unexpected. Without being bound by theory, it was possible that the cells did not tolerate the transfection with multiple copies of the GNAQ mutations. However, this much cell death with the transient transfection was not typically observed. Without intending to be bound by theory, it was also possible that puromycin was more toxic to cells with the mutations.

### Example 2: Cell Viability Assay Results Indicate that a R183Q Mutation in GNAQ Conferred Increased Sensitivity to Puromycin in HEK293 Cells

To determine whether mutations in GNAQ conferred increased sensitivity to puromycin, an MTT (3-(4,5-dimethylthiazol-2-yl)-2,5-diphenyltetrazolium bromide) assay dose response curve was done with HEK239 cells (including standard curve and normalization) after transient transfection. Puromycin exposure in HEK293 cells transiently transfected with R183Q or Q209L (no puromycin selection) over greater than a 100 fold dose range was then tested.

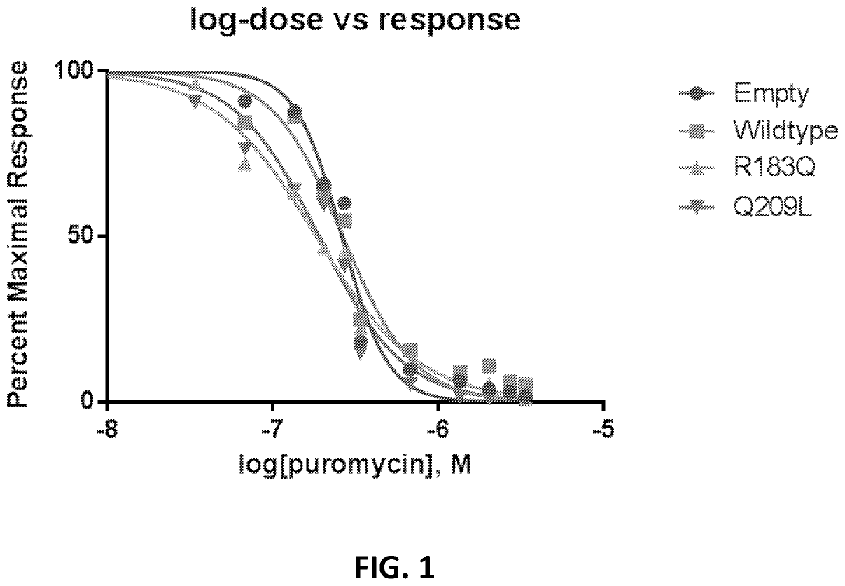

FIG. 1 shows the impact of puromycin upon cell survival, over a 100 fold range of concentrations. Results of the MTT assay showed that, compared to cells with the empty or WT constructs, cells with the mutant constructs had decreased survival after exposure to a lower dose. Half maximal inhibitory concentration (IC₅₀) for puromycin inhibition of cell growth was decreased in R183Q and Q209L mutant cells compared to cells with empty and wildtype constructs.

The assays were performed according to the methods described below.

### MTT Assay Methods

HEK 293 cells (5×10⁵ cells/well) in 6 well-plates were transient transfected with 1 μg of construct (pcDNA3.1-GNAQ, pcDAN3.1-R183Q and pcDNA3.1-Q209L) per well. Twenty-four (24) hours later, the cells were digested, counted and aliquoted into 24 well plates (0.8×10⁵ cells/well). After another 24 hours, the transfected cells were incubated with various concentrations of puromycin.

Three days later, the relative cell numbers were detected using MTT assay reagents (CellTiter 96® Aqueous One Solution Cell Proliferation Assay Reagents) as follows. After being thawed at room temperature, 40 μl the CellTiter 96® Aqueous One Solution Reagent was pipetted into each well of the 24-well assay plate containing the samples in 200 μl of culture medium. Then, the plates were incubated at 37° C. for 2 hours in a humidified, 5% CO₂ atmosphere. The supernatants were transferred into 96-well plates. The absorbance at 490 nm of each sample was read by using a plate reader (SpectraMax M5). Optical density directly correlated with viable cell quantity.

### Controls

Percent maximal response to puromycin (change in cell number) in the cells transfected with the R183Q mutation was compared to that in cells with the empty and those with the wildtype construct. Comparisons were made to that in cells with the other activating Q209L mutation (positive control).

### Statistical and Data Analyses

A software program (GraphPad Prism) was used to generate Dose Response Curves. A non-linear regression was done to graph the Log [puromycin molar concentration] versus Maximal Inhibitory Response. The GraphPad program was used to calculate the IC₅₀ of puromycin for each cell line (dose that gives a 50% maximal response) and the 95% confidence intervals. A 2-way ANOVA (Dose by Construct) was done to determine if there is a significant effect of construct upon puromycin effect with p-value of <0.05 used to determine significance.

### Example 3: Viability Assays to Confirm that Mutation R183Q in GNAQ in Various Cell Lines Cells Results in an Increased Sensitivity to Puromycin

To confirm that the R183Q mutation in GNAQ confers increased sensitivity to puromycin, the assay performed in Example 2 is repeated and additional cell viability assays are performed using various cell lines. HEK293T cells plated in 48 well plates are transiently transfected with empty, wildtype, R183Q, and Q209L (positive control) constructs and 24 hours later are exposed to puromycin for 1-4 days. A standard curve is first done to determine the range of cell numbers over which the assay is sensitive. All data is normalized to an internal experimental standard. Samples are analyzed on a 48 well plate reader (SpectraMax 5M) available to the lab by a researcher who is blinded to the construct and treatment identity of the samples. Ten (10) to twenty (20) different concentrations (including 0) of puromycin are tested in duplicate by MTT ((3-(4,5-dimethylthiazol-2-yl)-2,5-diphenyltetrazolium bromide)) assay in order to obtain a dose response curve plotting the % Maximal Response (change in cell number) versus the Log [drug concentration, in M] over a 100-fold concentration change. The dose response curve experiment is repeated a second time.

The MTT assay using EA.hy 926 endothelial cells, which are commercially available and widely used to study endothelial cell biology and function, are assayed as described above with the HEK293 cells. The same controls and experimental approach are also used to obtain a dose response curve for puromycin in these cells. The MTT assay may also be done with another commercially available endothelial cell line such as HUVEC cells.

### Example 4: Puromycin Vulnerability in GNAQ Mutants

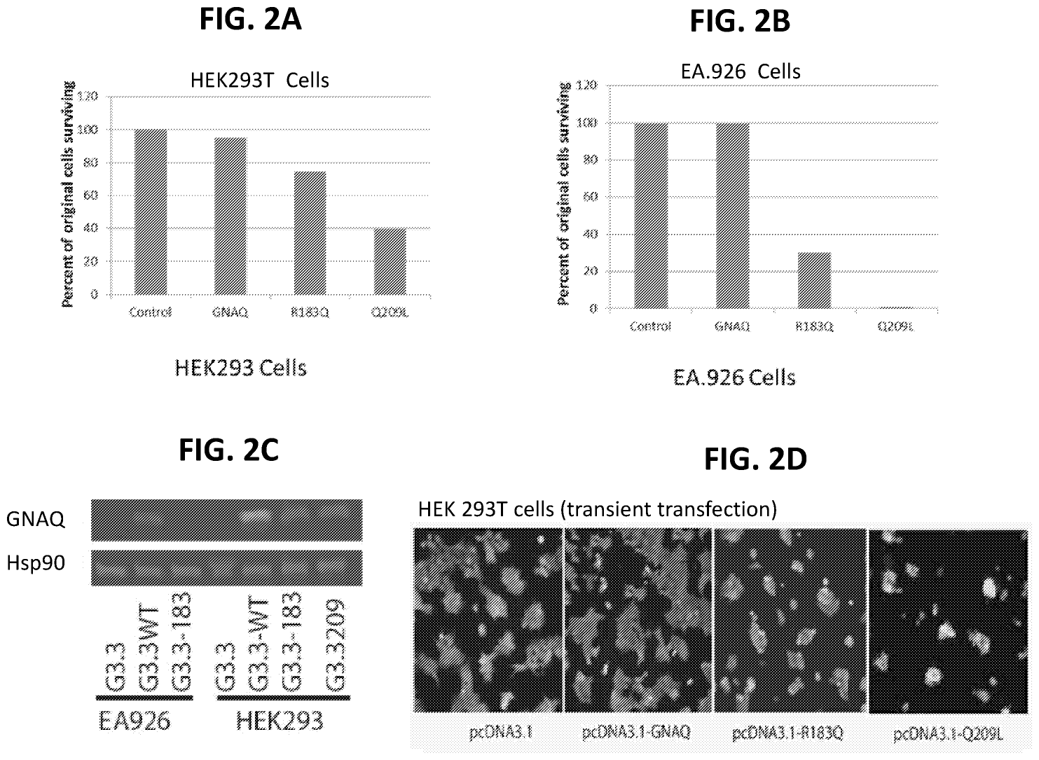

Puromycin vulnerability in GNAQ mutants was further investigated herein. pcDNA3.1-GNAQ, pcDNA3.1-R183Q, and GNAQ-Q209L were inserted into lentivirus-plasmids G3.3, which has Puromycin selection to generate G3.3-Gnaq, G3.3-R183Q, G3.3-Q209L (Comi lab). Then G3.3-Gnaq, G3.3-R183Q, G3.3-Q209L were used to produce lentivirus particles which were used to infect either HEK 293T cells or EA.hy926 cells. After being infected, the target cells were selected with 2 μg/ml puromycin (FIGS. 2A-2D). Only about 75% of the HEK 293T cells with the R183Q and only about 40% of the HEK 293T cells (FIG. 2A) with the Q209L mutation survived puromycin selection, and only about 30% of the EA.hy926 cells with the R183Q and less than 5% of the EA.hy926 cells with the Q209L mutation (FIG. 2B) survived the selection, whereas the stably transfected Empty and wild-type GNAQ cells demonstrated high survival. Furthermore, the mutants expressed lower levels of Gaq protein, while the cells with empty and wildtype constructs expressed higher levels of Gaq protein expression (FIG. 2C). Both HEK and endothelial cells with the mutant constructs grew more slowly than wildtype and demonstrated a more balled-up phenotype (FIG. 2D shows this morphology in transiently transfected cells; the endothelial cells with the Q209L mutation hardly grew at all.

Problems with the human mammary epithelial cell (HMEC) transfections (Table 1) were noted after failure to detect any induction of GNAQ by Western blots from most of the HMEC clones tested from infection 3. The clones were then examined for the presence of the full length GNAQ by PCR and only ˜25% of the clones contained full length plasmid-GNAQ. Of these few clones, stable, inducible GNAQ expression was seen only in 4 WT clones, 1-2 R183Q clones and 1-2 Q209L clones. The WT expression was always more robust than the mutant clones. Also, the morphology and growth patterns of the mutant clones, particularly of the Q209L clones, were noticeably different than the WT clones which themselves grew more slowly than the HMEC parent cells. These finding were utilized to modify ongoing attempts to establish stable endothelial cell lines using a Tet-ON system. With removal of puromycin from the media, these efforts which had previously resulted in non-viable cells or poor Gaq expression were quickly successful.

Overexpression of Gaq and p-ERK in HEK293T Cells Transiently Transfected with WT and Mutant GNAQ

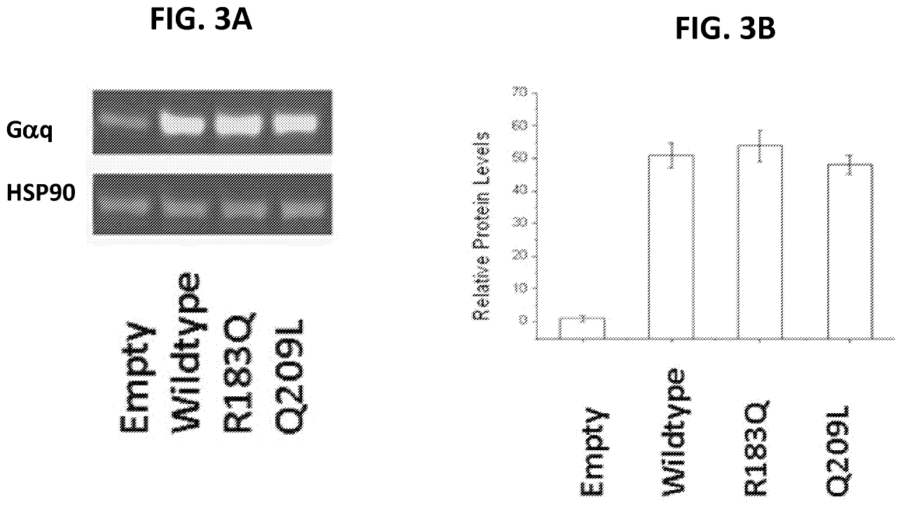

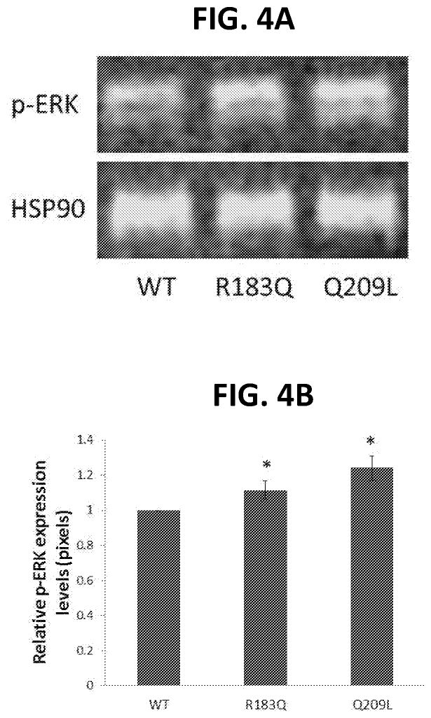

To assess the protein levels of Gaq in HEK293T cells transiently transfected with these plasmids, western blot analysis was performed (FIGS. 3A-3B) on the protein samples; data shown is from protein gathered for the 3 experiments for RNA and western analyses. The western blot analysis results demonstrated that protein levels of Gaq were significantly up-regulated (FIGS. 3A-3B, p<0.05) in HEK293T cells with WT or mutant constructs, compared to cells with the empty construct. p-ERK expression was also significantly increased in the mutants compared to WT (FIGS. 4A-4B).

### GNAQ Mutations are More Sensitive to Puromycin

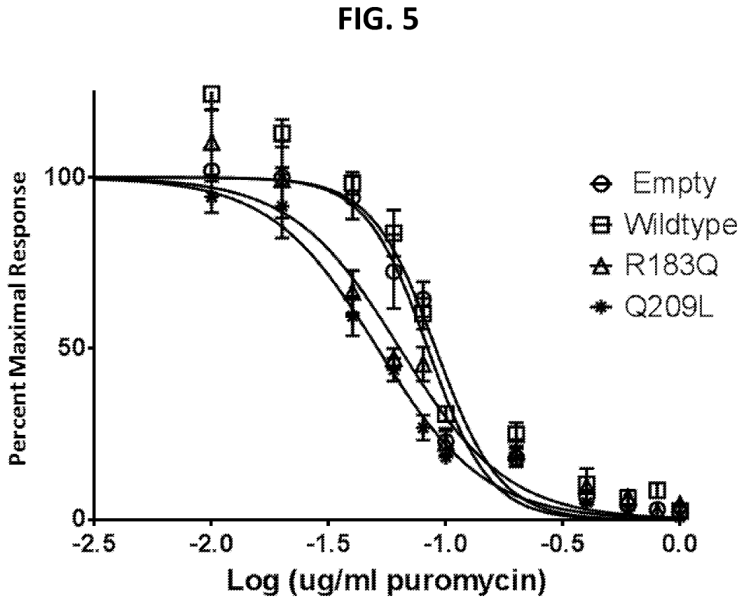

A dose response curve (cell number in response to puromycin concentration) for HEK293T cells transiently transfected pcDNA3.1-E, pcDNA3.1-GNAQ, pcDNA3.1-R183Q and pcDNA3.1-Q209L and treated with puromycin is shown in FIG. 5. Cells expressing either the pcDNA3.1-R183Q or pcDNA3.1-Q209L plasmids were more sensitive to puromycin compared with cells transfected with the pcDNA3.1-E or pcDNA3.1-GNAQ plasmids. The half maximal inhibitory concentration (IC₅₀) of puromycin for cells with the pcDNA3.1-R183Q was 0.064 μg/ml (95% CI 0.053 to 0.077 μg/ml) and for pcDNA3.1-Q209L was 0.051 μg/ml (95% CI 0.046 to 0.058 μg/ml) while for cells with pcDNA3.1-E it was 0.084 μg/ml (95% CI 0.074 to 0.095 μg/ml) and for the cells with the pcDNA3.1-GNAQ it was 0.091 μg/ml (95% CI 0.072 to 0.114 μg/ml).

### Real Time PCR Results

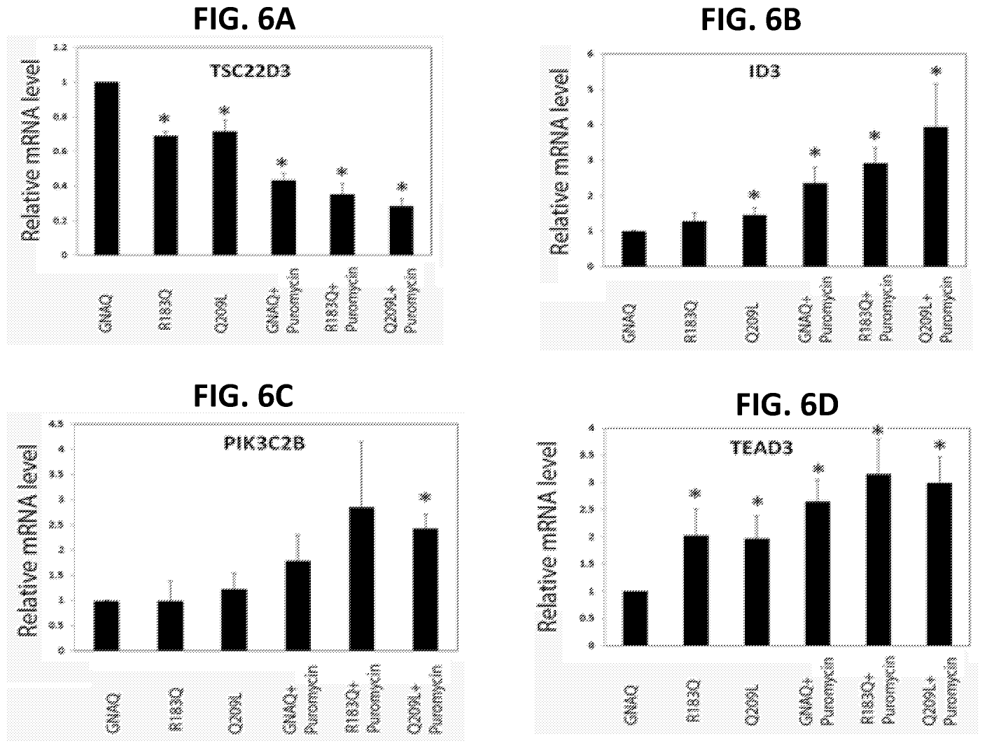

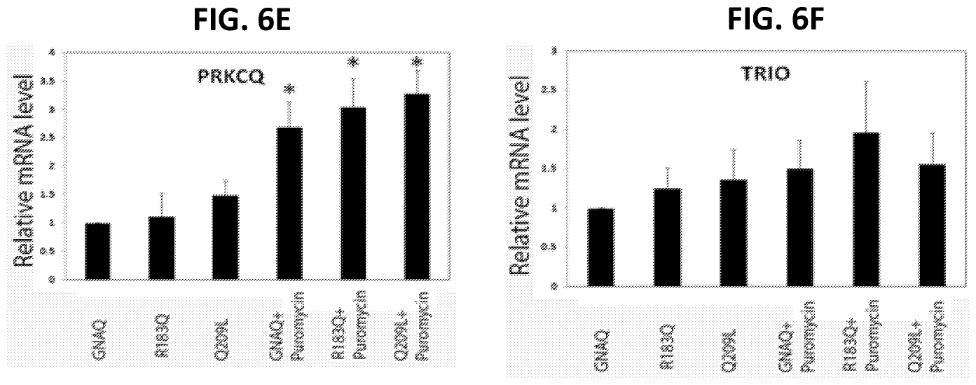

A panel of genes, important to the regulation of the pathways downstream of GNAQ, were evaluated by RT-PCR in mRNA samples gathered from cells with the pcDNA3.1-E, pcDNA3.1-GNAQ, pcDNA3.1-R183Q or pcDNA3.1-Q209L plasmids with or without puromycin treatment of 0.04 μg/μl (FIGS. 6A-6F, n=3 per group from 3 separate experiments). In cells expressing either R183Q or Q209L, the mRNA levels of TSC22D3 were down-regulated compared to empty or wildtype plasmid (vehicle treated). Treatment with puromycin was associated with decreased TSC22D3 mRNA expression (compared to vehicle treated wildtype), while treatment of cells expressing the R183Q or Q209L plasmids was associated with further decreased mRNA levels of TSC22D3 (FIG. 6A). mRNA levels of ID3 was up-regulated in cells overexpressing Q209L, and was also up-regulated in wildtype cells treated by puromycin (compared to vehicle treated). ID3 was also upregulated in cells expressing either 183Q and Q209L mutant Gaq treated with puromycin (compared to wildtype puromycin treated and to vehicle treated mutant cells; FIG. 6B).

Furthermore, both R183Q and Q209L mutants were associated with significantly up-regulated mRNA levels of TEAD3 (P-value<0.05) compared to HEK293T cells with wildtype construct (FIG. 6D). PIK3C2B (FIG. 6C), PRKCQ (FIG. 6E), and TRIO (FIG. 6F) levels were increased compared to wildtype but not significantly. Levels of these transcripts were up-regulated in all three constructs by puromycin alone, but only significantly for TEAD3 and PRKCQ (FIG. 6E), and for PIK3C2B in Q209L cells treated with puromycin (FIG. 6C).

### Western Results

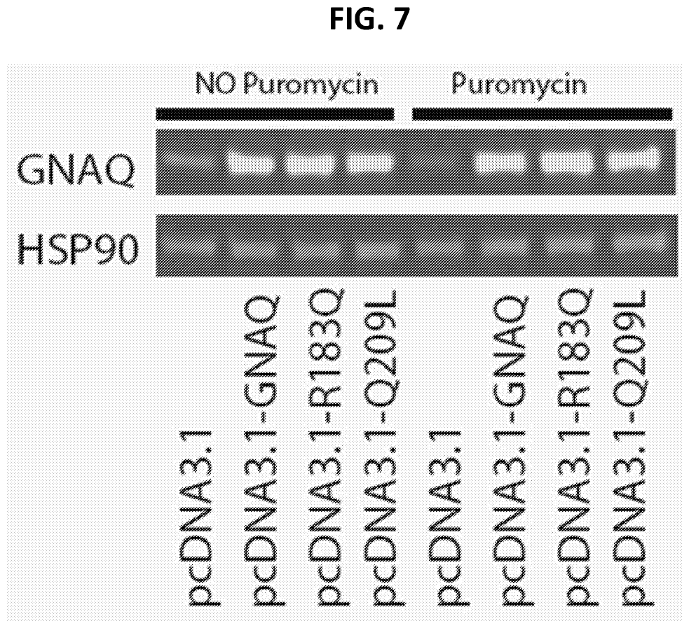

No significant differences were observed in Gaq levels between the puromycin and vehicle treated cells, and no difference in molecular weight of Gaq was noted in any of the cells treated with puromycin, either wildtype or empty (FIG. 7). Protein levels of ID3 in R183Q and Q209L cells further increased with puromycin treatment in all three groups (pcDNA3.1-GNAQ, pcDNA3.1-R183Q or pcDNA3.1-Q209L).

### Immunohistochemistry Results

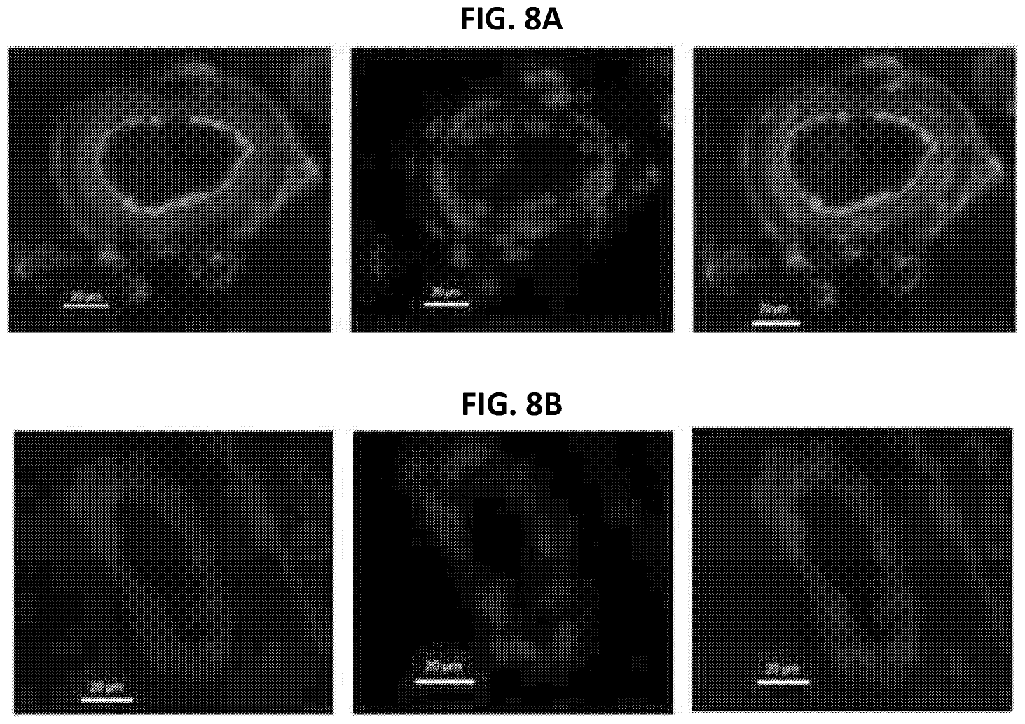

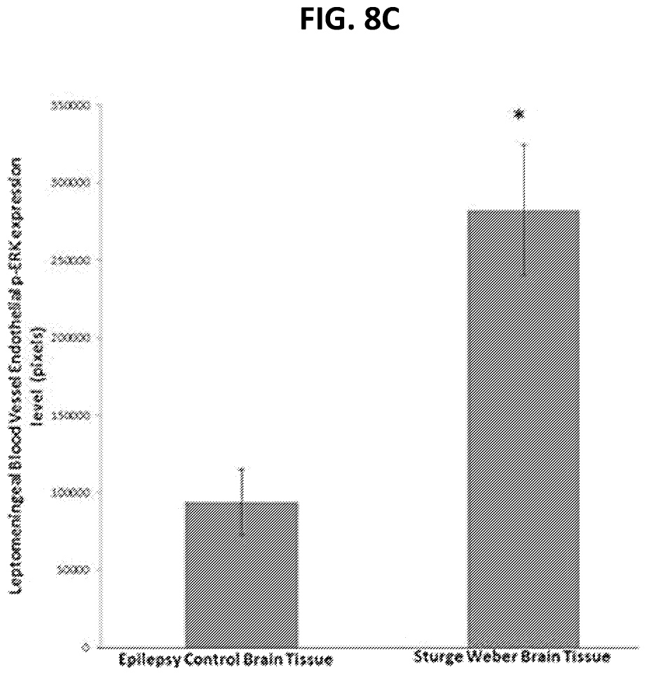

Disrupted organization of blood vessel cellular structure was noted on a-tubulin immunohistochemistry, and discontinuity of CD34+labeling of SWS leptomeningeal blood vessels compared to vessels from epilepsy controls was noted. Blood vessels in Sturge-Weber brain tissue samples had significantly higher p-ERK expression in the endothelial cells of leptomeningeal vessels than epilepsy control samples (28,2192±SEM16,8902 vs. 9,4042±SEM 4,7340, p<0.05 (FIGS. 8A-8C).

Results described herein were obtained using the following methods and materials.

### GNAQ and Mutant Plasmids

The plasmids of wild type GNAQ its mutations (pcDNA3.1-GNAQ, pcDAN3.1-R183Q and pcDNA3.1-Q209L) were generously provided by Dr. Kun-Liang Guan (Department of Pharmacology, University of California San Diego, USA). The empty plasmid (pcDNA3.1E) was constructed from pcDNA3.1-GNAQ by cutting the cDNA of GNAQ out with Pmel, and then the backbone of pcDNA3.1 was ligated. All GNAQ WT and mutant plasmids (pcDNA3.1-R183Q, pcDNA3.1-Q209L) were sequenced with CMV promoter as forward primer and BGH sequence as reverse primer and the correct mutation sites were confirmed. Sequence results are provided below:

GNAQ Wild type forward
(SEQ ID NO: 10)
NNNNNNNNNNNNNNNNNNGAGCTCTCTGGCTAACTAGAGAACCCACTGC
TTACTGGCTTATCGAAATTAATACGACTCACTATAGGGAGACCCAAGCT
GGCTAGCGTTTAAACTTAAGCTTGGTACCACCATGACTCTGGAGTCCAT
CATGGCGTGCTGCCTGAGCGAGGAGGCCAAGGAAGCCCGGCGGATCAAC
GACGAGATCGAGCGGCAGCTCCGCAGGGACAAGCGGGACGCCCGCCGGG
AGCTCAAGCTGCTGCTGCTCGGGACAGGAGAGAGTGGCAAGAGTACGTT
TATCAAGCAGATGAGAATCATCCATGGGTCAGGATACTCTGATGAAGAT
AAAAGGGGCTTCACCAAGCTGGTGTATCAGAACATCTTCACGGCCATGC
AGGCCATGATCAGAGCCATGGACACACTCAAGATCCCATACAAGTATGA
GCACAATAAGGCTCATGCACAATTAGTTCGAGAAGTTGATGTGGAGAAG
GTGTCTGCTTTTGAGAATCCATATGTAGATGCAATAAAGAGTTTATGGA
ATGATCCTGGAATCCAGGAATGCTATGATAGACGACGAGAATATCAATT
ATCTGACTCTACCAAATACTATCTTAATGACTTGGACCGCGTAGCTGAC
CCTGCCTACCTGCCTACGCAACAAGATGTGCTTAGAGTTCGAGTCCCCA
CCACAGGGATCATCGAATACCCCTTTGACTTACAAAGTGTCATTTTCAG
AATGGTCGATGTAGGGGGCCAAAGGTCAGAGAGAAGAAAATGGATACAC
TGCTTTGAAAATGTCACCTCTATCATGTTTCTAGTAGCGCTTAGTGAAT
ATGATCAAGTTCTCGTGGAGTCAGACAATGAGAACCGAATGGAGGAAAG
CAAGGCTCTCTTTAGAACAATTATCACATACCCCTGGTTCCAGAACTCC
TCGGTTATTCTGTTCTTAAACAAGAAAGATCTTCTAGAGGAGAAAATCA
TGTATTCCCATCTAGTCGACTACTTCCCAGAATATGATGGACCCCAGAG
AGATGCCCAGGCAGCCCGAGA
Wild type reverse
(SEQ ID NO: 11)
GGCGGATCAACGACGAGATCGAGCGGCAGCTCCGCAGGGACAAGCGGGAC
GCCCGCCGGGAGCTCAAGCTGCTGCTGCTCGGGACAGGAGAGAGTGGCAA
GAGTACGTTTATCAAGCAGATGAGAATCATCCATGGGTCAGGATACTCTG
ATGAAGATAAAAGGGGCTTCACCAAGCTGGTGTATCAGAACATCTTCACG
GCCATGCAGGCCATGATCAGAGCCATGGACACACTCAAGATCCCATACAA
GTATGAGCACAATAAGGCTCATGCACAATTAGTTCGAGAAGTTGATGTGG
AGAAGGTGTCTGCTTTTGAGAATCCATATGTAGATGCAATAAAGAGTTTA
TGGAATGATCCTGGAATCCAGGAATGCTATGATAGACGACGAGAATATCA
ATTATCTGACTCTACCAAATACTATCTTAATGACTTGGACCGCGTAGCTG
ACCCTGCCTACCTGCCTACGCAACAAGATGTGCTTAGAGTTCGAGTCCCC
ACCACAGGGATCATCGAATACCCCTTTGACTTACAAAGTGTCATTTTCAG
AATGGTCGATGTAGGGGGCCAAAGGTCAGAGAGAAGAAAATGGATACACT
GCTTTGAAAATGTCACCTCTATCATGTTTCTAGTAGCGCTTAGTGAATAT
GATCAAGTTCTCGTGGAGTCAGACAATGAGAACCGAATGGAGGAAAGCAA
GGCTCTCTTTAGAACAATTATCACATACCCCTGGTTCCAGAACTCCTCGG
TTATTCTGTTCTTAAACAAGAAAGATCTTCTAGAGGAGAAAATCATGTAT
TCCCATCTAGTCGACTACTTCCCAGAATATGATGGACCCCAGAGAGATGC
CCAGGCAGCCCGAGAATTCATTCTGAAGATGTTCGTGGACCTGAACCCAG
ACAGTGACAAAATTATCTACTCCCACTTCACGTGCGCCACAGACACCGAG
AATATCCGCTTTGTCTTTGCTGCCGTCAAGGACACCATCCTCCAGTTGAA
CCTGAAGGAGTACAATCTGGTCTAACTCGAGTCTAGNGNNNNNNNNNNNN
183 Forward
(SEQ ID NO: 12)
NNNNNNNNNNNNNCTTNNNCTTGGTACCNCCATGACTCTGGAGTCCATCA
TGGCGTGCTGCCTGAGCGAGGAGGCCAAGGAAGCCCGGCGGATCAACGAC
GAGATCGAGCGGCAGCTCCGCAGGGACAAGCGGGACGCCCGCCGGGAGCT
CAAGCTGCTGCTGCTCGGGACAGGAGAGAGTGGCAAGAGTACGTTTATCA
AGCAGATGAGAATCATCCATGGGTCAGGATACTCTGATGAAGATAAAAGG
GGCTTCACCAAGCTGGTGTATCAGAACATCTTCACGGCCATGCAGGCCAT
GATCAGAGCCATGGACACACTCAAGATCCCATACAAGTATGAGCACAATA
AGGCTCATGCACAATTAGTTCGAGAAGTTGATGTGGAGAAGGTGTCTGCT
TTTGAGAATCCATATGTAGATGCAATAAAGAGTTTATGGAATGATCCTGG
AATCCAGGAATGCTATGATAGACGACGAGAATATCAATTATCTGACTCTA
CCAAATACTATCTTAATGACTTGGACCGCGTAGCTGACCCTGCCTACCTG
CCTACGCAACAAGATGTGCTTAGAGTTCAAGTCCCCACCACAGGGATCAT
CGAATACCCCTTTGACTTACAAAGTGTCATTTTCAGAATGGTCGATGTAG
GGGGCCAAAGGTCAGAGAGAAGAAAATGGATACACTGCTTTGAAAATGTC
ACCTCTATCATGTTTCTAGTAGCGCTTAGTGAATATGATCAAGTTCTCGT
GGAGTCAGACAATGAGAACCGAATGGAGGAAAGCAAGGCTCTCTTTAGAA
CAATTATCACATACCCCTGGTTCCAGAACTCCTCGGTTATTCTGTTCTTA
AACAAGAAAGATCTTCTAGAGGAGAAAATCATGTATTCCCATCTAGTCGA
CTACTTCCCAGAATATGATGGACCCCAGAGAGATGCCCAGGCAGCCCGAG
AATTCATTCTGAAGATGTTCGTGGACCTGAACCCAGACAGTGACAAAATT
ATCTACTCCCACTTCACGTGCGCCACAGACACCGAGAATATCCGCTTTGT
183 Reverse
(SEQ ID NO: 13)
GATCAACGACGAGATCGAGCGGCAGCTCCGCAGGGACAAGCGGGACGCCC
GCCGGGAGCTCAAGCTGCTGCTGCTCGGGACAGGAGAGAGTGGCAAGAGT
ACGTTTATCAAGCAGATGAGAATCATCCATGGGTCAGGATACTCTGATGA
AGATAAAAGGGGCTTCACCAAGCTGGTGTATCAGAACATCTTCACGGCCA
TGCAGGCCATGATCAGAGCCATGGACACACTCAAGATCCCATACAAGTAT
GAGCACAATAAGGCTCATGCACAATTAGTTCGAGAAGTTGATGTGGAGAA
GGTGTCTGCTTTTGAGAATCCATATGTAGATGCAATAAAGAGTTTATGGA
ATGATCCTGGAATCCAGGAATGCTATGATAGACGACGAGAATATCAATTA
TCTGACTCTACCAAATACTATCTTAATGACTTGGACCGCGTAGCTGACCC
TGCCTACCTGCCTACGCAACAAGATGTGCTTAGAGTTCAAGTCCCCACCA
CAGGGATCATCGAATACCCCTTTGACTTACAAAGTGTCATTTTCAGAATG
GTCGATGTAGGGGGCCAAAGGTCAGAGAGAAGAAAATGGATACACTGCTT
TGAAAATGTCACCTCTATCATGTTTCTAGTAGCGCTTAGTGAATATGATC
AAGTTCTCGTGGAGTCAGACAATGAGAACCGAATGGAGGAAAGCAAGGCT
CTCTTTAGAACAATTATCACATACCCCTGGTTCCAGAACTCCTCGGTTAT
TCTGTTCTTAAACAAGAAAGATCTTCTAGAGGAGAAAATCATGTATTCCC
ATCTAGTCGACTACTTCCCAGAATATGATGGACCCCAGAGAGATGCCCAG
GCAGCCCGAGAATTCATTCTGAAGATGTTCGTGGACCTGAACCCAGACAG
TGACAAAATTATCTACTCCCACTTCACGTGCGCCACAGACACCGAGAATA
TCCGCTTTGTCTTTGCTGCCGTCAAGGACACCATCCTCCAGTTGAACCTG
AAGGAGTACAATCTGGTCTAACNNGANNNNNNNNNNNNNNNNNNNNNNNC
209 Forward
(SEQ ID NO: 14)
NNNNNNNNNNNNNNNNNNNNANCTCTCTGGCTANCTAGAGAACCCACTGC
TTACTGGCTTATCGAAATTAATACGACTCACTATAGGGAGACCCAAGCTG
GCTAGCGTTTAAACTTAAGCTTGGTACCACCATGACTCTGGAGTCCATCA
TGGCGTGCTGCCTGAGCGAGGAGGCCAAGGAAGCCCGGCGGATCAACGACG
AGATCGAGCGGCAGCTCCGCAGGGACAAGCGGGACGCCCGCCGGGAGCTCA
AGCTGCTGCTGCTCGGGACAGGAGAGAGTGGCAAGAGTACGTTTATCAAGC
AGATGAGAATCATCCATGGGTCAGGATACTCTGATGAAGATAAAAGGGGCT
TCACCAAGCTGGTGTATCAGAACATCTTCACGGCCATGCAGGCCATGATCA
GAGCCATGGACACACTCAAGATCCCATACAAGTATGAGCACAATAAGGCTC
ATGCACAATTAGTTCGAGAAGTTGATGTGGAGAAGGTGTCTGCTTTTGAGA
ATCCATATGTAGATGCAATAAAGAGTTTATGGAATGATCCTGGAATCCAGG
AATGCTATGATAGACGACGAGAATATCAATTATCTGACTCTACCAAATACT
ATCTTAATGACTTGGACCGCGTAGCTGACCCTGCCTACCTGCCTACGCAAC
AAAGATGTGCTTAGAGTTCGAGTCCCCACCCAGGGATCATCGAATACCCCT
TTGACTTACAAAGTGTCATTTTCAGAATGGTCGATGTAGGGGGCCTAAGGT
CAGAGAGAAGAAAATGGATACACTGCTTTGAAAATGTCACCTCTATCATGT
TTCTAGTAGCGCTTAGTGAATATGATCAAGTTCTCGTGGAGTCAGACAATG
AGAACCGAATGGAGGAAAGCAAGGCTCTCTTTAGAACAATTATCACATACC
CCTGGTTCCAGAACTCCTCGGTTATTCTGTTCTTAAACAAGAAAGATCTTC
TAGAGGAGAAAATCATGTATTCCCATCTAGTCGACTACTTCCCAGAATATG
ATGGACCCCAGAGAGATGCCCAGGCAGCCCGAGA
209 Reverse
(SEQ ID NO: 15)
GGCGGATCAACGACGAGATCGAGCGGCAGCTCCGCAGGGACAAGCGGGAC
GCCCGCCGGGAGCTCAAGCTGCTGCTGCTCGGGACAGGAGAGAGTGGCAA
GTGAGTACTTATCAAGCAGATGAGAATCATCCATGGGTCAGGATACTCTG
ATGAAGATAAAAGGGGCTTCACCAAGCTGGTGTATCAGAACATCTTCACG
GCCATGCAGGCCATGATCAGAGCCATGGACACACTCAAGATCCCATACAA
GTATGAGCACAATAAGGCTCATGCACAATTAGTTCGAGAAGTTGATGTGG
AGAAGGTGTCTGCTTTTGAGAATCCATATGTAGATGCAATAAAGAGTTTA
TGGAATGATCCTGGAATCCAGGAATGCTATGATAGACGACGAGAATATCA
ATTATCTGACTCTACCAAATACTATCTTAATGACTTGGACCGCGTAGCTG
ACCCTGCCTACCTGCCTACGCAACAAGATGTGCTTAGAGTTCGAGTCCCC
ACCACAGGGATCATCGAATACCCCTTTGACTTACAAAGTGTCATTTTCAG
AATGGTCGATGTAGGGGGCCTAAGGTCAGAGAGAAGAAAATGGATACACT
GCTTTGAAAATGTCACCTCTATCATGTTTCTAGTAGCGCTTAGTGAATAT
GATCAAGTTCTCGTGGAGTCAGACAATGAGAACCGAATGGAGGAAAGCAA
GGCTCTCTTTAGAACAATTATCACATACCCCTGGTTCCAGAACTCCTCGG
TTATTCTGTTCTTAAACAAGAAAGATCTTCTAGAGGAGAAAATCATGTAT
TCCCATCTAGTCGACTACTTCCCAGAATATGATGGACCCCAGAGAGATGC
CCAGGCAGCCCGAGAATTCATTCTGAAGATGTTCGTGGACCTGAACCCAG
ACAGTGACAAAATTATCTACTCCCACTTCACGTGCGCCACAGACACCGAG
AATATCCGCTTTGTCTTTGCTGCCGTCAAGGACACCATCCTCCAGTTGAA
CCTGAAGGAGTACAATCGAGTCTAGNGNNCCNNNNNNN

### Stable Transfection Experiments

Confluent GP2-293T cells in T75 flasks were transfected using LIPOFECTAMINE® transfection reagent with packaging vector (VSV-G) and plasmid containing either WT, R183Q or Q209L GNAQ. Supernatant containing virus was collected 48 hours later and filtered. HMECs, at ˜50% confluent, were infected with viral supernatant and polybrene (Sigma-Aldrich Al-118) and then re-infected 24 hours later for a total infection time of 48 hours. Cells were grown to confluency then split 1:3. Puromycin was added at this point to select for transformed cells. The dosage was determined by a dose response curve performed in untransformed HMECs. 1 μg/ml killed 100% of the cells after 48 hours.

### Transient Transfection

HEK293T cells (ATCC) (5×10⁵/well) plated in 6 well plates, which had been coated with poly-L-lysine (Sigma-Aldrich, P8920), were incubated at 37° C., 5% CO₂ overnight. On the second day, the plasmids of pcDNA3.1E, pcDNA3.1-GNAQ, pcDNA3.1-R183Q and pcDNA3.1-Q209L were transiently transfected into the HEK293T cells by using FUGENE 6® transfection reagent. For each plasmid, 1 μg plasmid DNA was added into 100 μl serum-free OPTI-MEM. 6 μl FUGENE 6® transfection reagent was added into 100 μl serum-free OPTI-MEM® medium. After being incubated for 5 minutes at room temperature, the diluted DNA and diluted FUGENE 6® transfection reagent were combined together and were incubated in hood for 15 min. Then, the DNA: FUGENE 6® transfection reagent mixture was added into HEK293T cells cultured in 2 ml of Dulbecco's Modified Eagle Medium (DMEM) without antibiotics. Plates were swirled to disperse mixture evenly. After being incubated at 37° C., 5% CO₂ for 24 hours, the transiently transfected cells were fed with 2 ml of fresh media.

### Western Blot Assay

Treated cells were harvested with ice-cold radioimmunoprecipitation assay (RIPA) buffer (150 mM NaCl, 1.0% IGEPAL® CA-630, 0.5% sodium deoxycholate, 0.1% SDS, and 50 mM Tris, pH 8.0) (Sigma-Aldrich) plus phosphatase inhibitor cocktail (Cell signaling technology, #5870) and analyzed by western blot as follows: Quantitative protein samples were denatured in 4× LDS Sample buffer (Invitrogen) at 100° C. for 6 minutes. Samples were then subjected to SDS-PAGE by using Bio-Rad 4-15% gradient gels and transferred to PVDF membrane. The membranes were blocked with Li-cor ODYSSEY® Blocking buffer for 30 minutes at room temperature. Then the membranes were incubated with primary antibody (GaQ 1:125, Santa Cruz Biotechnology, sc-393; p-ERK 1:1000, Cell Signaling Technology, 4370; ERK 1:1000, Cell signaling Technology, 4696; HSP90 1:1000, Cell Signaling Technology, 4877) in a 1:1 solution of Li-cor ODYSSEY® blocking buffer and Tris-Buffered Saline and 0.1% Tween 20 (TBST) overnight at 4° C. The membranes were then washed three times for 10 min each in Tris-Buffered Saline and 0.1% Tween 20 (TBST) then probed with goat anti-mouse (IR-Dye 680RD) or goat anti Rabbit (IR-Dye-800CW) labeled secondary antibody in 1:1 Li-cor ODYSSEY® blocking buffer to TBST for 1 hour at room temperature. After being washed three times with TBST, the membranes were imaged using a Li-cor ODYSSEY® scanner. Bands were quantified using Image J software program.

### MIT Assay

HEK293T cells (5×10⁵ cells/well) in 6 well-plates were transiently transfected with 1 μg (pcDNA3.1-E, pcDNA3.1-GNAQ, pcDAN3.1-R183Q and pcDNA3.1-Q209L) per well using FUGENE 6®. Twenty-four (24) hours later, the cells were digested, counted and aliquoted into 96-well plates (1×10⁴ cells/well). After another 24 hours, the transfected cells were incubated with a 100-fold concentration series of puromycin. Three days later, the relative cell numbers were detected using CELLTITER 96® Aqueous One Solution Cell Proliferation Assay Reagents (Promega #G3582) as follows: After being thawed at room temperature, 10 μl of CELLTITER 96® Aqueous One Solution Reagent was pipet into each well of the 96-well assay plate containing the samples in 100 μl of culture medium. Then, the plates were incubated at 37° C. for 2 hours in a humidified, 5% CO₂ atmosphere. The absorbance at 490 nm of each sample was read by using SpectraMAX M5. Readings were normalized to that in pcDNA3.1-GNAQ cells receiving vehicle. Results were analyzed in GraphPad. Experiment was done in quadruplicate samples.

### RNA Isolation

HEK293T cells (5×10⁵ cells/well) were transfected with 1 μg plasmid (pcDNA3.1-GNAQ, pcDAN3.1-R183Q or pcDNA3.1-Q209L) in 6-well plates. Twenty-four (24) hours later, cells were digested and re-plated into 6-well plates. Twenty-four (24) hours later, the plates were incubated in DMEM (10% FBS) with or without 0.04 μg/ml puromycin. Three days later, total RNA of treated HEK293T cells were isolated by using RNEASY® Mini Kit (QIAGEN) as follows. Media was aspirated, and the cells washed twice with PBS. 350 μl Buffer RLT with B-Mercaptoethanol was added to the each well. The lysate was pipeted into a microcentrifuge tube and pipeted to mix. 1 volume of 70% ethanol was added to the lysate. Lysate was transferred to an RNEASY® spin column placed in a 2 ml collection tube (supplied) and centrifuged. RNEASY® spin column was washed as per kit instructions. 50 μl RNase-free water was added directly to the center of the spin column membrane which was centrifuged for 1 min at 12,000 rpm to elute the RNA. Three separate experiments were performed to obtain triplicate mRNA and protein samples for analysis.

### Reverse Transcription

Reverse Transcription was carried out with the using the High Capacity cDNA Reverse Transcription Kits and based on Applied Biosystems' protocol. 2×RT master mix is prepared using the kit components before preparing the reaction plate and kit components allowed to thaw on ice. Referring to one reaction amount of components, the volume of components needed to prepare the required number of reactions was calculated. Then 10 μl of 2× RT master mixes was pipetted into each well of 500 μl PCR clean tubes. 10 μL of RNA sample (1 μg) was pipetted into each tube which is then mixed, centrifuged and then placed in a thermal cycler with the program of (10 min at 25° C., 120 min at 37° C., 5 min at 85° C., then 4° C.). After the thermal cycler, 1 μl RNase H was added into the tubes and incubated at 37° C. for 20 min. Finally, 600 μl Nuclease-free H₂O was added into each tube.

### Real Time PCR

The primers for Real-Time PCR were designed online with Primer-BLAST software tool. For each gene, at least three primers at different locations were designed based on the cDNA of the genes. The primer concentrations were normalized and were adjusted to 5 pmol/μl. The real-time PCR reaction mixture of 20 μl included 10 μl SYBR Green Mix (2×), 2 μl primer pair mix, and 8 μl diluted cDNA solution. The Real-time PCR of loaded samples were run on CFX connect™ real-time PCR system (Bio-rad) with extension steps of (50° C. 2 min 1 cycle, 95° C. 10 min 1 cycle, 95° C. 15 sec then 60° C. 1 min 40 cycles, 72° C. 10 min, 1 cycle) followed by a melting curve analysis (from 55° C. to 95° C. increments 0.05° C./Sec) to guarantee absence of non-specific amplification. The primers with a unique peak in melting curve were chosen for next step. The primers were also only used for next experiments after they were confirmed by obtaining the same results from other primers for the same gene. With the final confirmed primers, the RNA levels in triplicate samples, obtained from three separate experiments, were measured.

### Immunohistochemistry

Series of slides from Sturge-Weber Syndrome fixed brain tissue and surgical epilepsy focal cortical dysplasia disease fixed control samples were deparaffinized with HemoDe solvent and rehydrated with ethanol. Antigen retrieval was then done for an hour at steaming temperature in 1X Citrate buffer. Slides were cooled, washed in TBS, blocked for nonspecific reactivity and then stained for CD34 and alpha-tubulin. The slides were incubated overnight at 4° C. in the primary antibodies; anti CD34 (rabbit monoclonal, 1:1000, Abcam Inc., Cambridge, MA, Cat #ab81289) and alpha-tubulin (mouse monoclonal, 1:1000; Santa Cruz Biotechnology, Cat #sc-23948). The secondary antibodies were applied the next day; Alexa 594 (1:500; Invitrogen, Carlsbad, CA) for cells marked with CD34, and Alexa 488 (1:500; Invitrogen, Carlsbad, CA) for alpha-tubulin detection. Slides were coverslipped with prolong antifade medium with DAPI (Cell Signaling). Intact tissue (determine by robust alpha-tubulin staining and CD34 staining) were stained (adjacent sections) for p-ERK and total ERK using rabbit monoclonal Phospho-p44/42 MAPK (p-ERK, 1:100, Cell Signaling Technology, Cat #4370) and mouse p44/42 MAPK (Total ERK, 1:300, Cell Signaling Technology, Cat #4696). The secondary antibodies applied were Alexa 594 (1:500; Invitrogen, Carlsbad, CA) for cells marked with p-ERK, and Alexa 488 (1:500; Invitrogen, Carlsbad, CA) for total ERK detection.

p-ERK intensity levels in the endothelial cells of blood vessels in the leptomeninges were visualized using the AxioVision Apotome System microscope and software (Carl Zeiss MicroImaging). Representative images from the greatest p-ERK labeling seen in that brain section (2 fields of view/sample) were taken at 20× using standardized camera settings. Image J software was used to determine the average intensity density of all leptomeningeal vessels wholly contained within the field of view captured in each image. Each cross-sectional blood vessel image was traced on the inside of the blood vessel (inner most ring of the vessel) and was also traced on the outer aspect of the cross-sectional endothelial layer. The average intensity density of p-ERK labeling for the endothelial cell layer was measured by Image J software analysis. Student's t-test was used to compare the average intensity density in the SWS leptomeningeal vessels versus the epilepsy control leptomeningeal vessels±SEM.

TABLE 1
Results of initial induction studies with
human mammary epithelial cells (HMEC)

| In- | Dosage of | | Dox | GNAQ | |
|---|---|---|---|---|---|
| fection | Puromycin | Clones | induced | expressing | Observations |
| 1 | 1 ug/ml | 0 | | | |
| 2 | 1 ug/ml | 0 | | | |
| 3 | 0.25 ug/ml, | ~50 of | WT-16 | WT-1 | Morphology |
| | 0.33 ug/ml, | each (WT, | RQ-17 | RQ-1 | of mutants |
| | 1 ug/ml, | RQ, QL) | QL-17 | QL-1 | different and |
| | 0.5 ug/ml | | | | they grew |
| | | | | | more slowly |
| | | | | | than WT |
| 4 | 1 ug/ml | WT-16 | | | |
| | | RQ-12 | | | |
| | | QL-16 | | | |
| 5 | 1 ug/ml | 0 | | | |
| 6 | 1 ug/ml | WT-15 | WT-12 | WT-3 | Morphology |
| | | RQ-9 | RQ-5 | RQ-1 | of mutants |
| | | QL-12 | QL-9 | QL-1 | different and |
| | | | | | they grew |
| | | | | | more slowly |
| | | | | | than WT |

TABLE 2
RT-PCR primer sequences

| | | | SEQ ID |
|---|---|---|---|
| Name | | Sequence | NO: |
| ID3 | Forward 1 | AGGTCACTGTAGCGGACTTC | 16 |
| ID3 | Reverse 1 | CTTCATGCTGGGGAGTGAGT | 17 |
| TSC22D3 | Forward 1 | TCTGTTTCGTGAAGGCAGGG | 18 |
| TSC22D3 | Reverse 1 | TGTAATCCCACACTGGGCTG | 19 |
| TEAD3 | Forward 3 | TTGTGTACCGTATCCACCGC | 20 |
| TEAD3 | Reverse 3 | TTCTCCAGCACGCTGTTCAT | 21 |
| TRIO | Forward 3 | GCACCATGTCCTGGATGTCA | 22 |
| TRIO | Reverse 3 | CCTGCTGGAAAACACACAGC | 23 |
| PRKCQ | Forward 4 | TCCAACTTTGACTGCGGGTC | 24 |
| PRKCQ | Reverse 4 | ACATCTGCCCGTTCTCTGAT | 25 |
| TAF15 | Forward 3 | ATGGAAATCCAGGCAGCCAA | 26 |
| TAF15 | Reverse 3 | TGTCCATAACTGGAGTAACCGC | 27 |
| PIK3C2B | Forward 1 | TTTGTGCTTTGGGGAGCAGA | 28 |
| PIK3C2B | Reverse 1 | GCTAAGGCTTCCTTCAGCCA | 29 |

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method of inhibiting proliferation and/or reducing survival of a cell comprising a GNAQ polynucleotide or polypeptide having a R183Q or Q209L mutation, the method comprising contacting the cell with puromycin or a puromycin analog, thereby inhibiting proliferation and/or reducing survival of the cell.

2. A method of reducing a vascular malformation in a subject, the method comprising administering to the subject an effective amount of puromycin or a puromycin analog.

3. A method of inhibiting progression of a vascular malformation in a subject, the method comprising administering to the subject an effective amount of puromycin or a puromycin analog.

4. A method of reducing appearance of a birthmark in a subject, the method comprising administering to the subject an effective amount of puromycin or a puromycin analog.

5. A method of treating a vascular malformation or related condition in a subject, the method comprising administering to the subject an effective amount of puromycin or puromycin analog.

6. The method of any of claims 2-5, further comprising administering laser treatment to the subject.

7. A method of treating a uveal melanoma in a subject, the method comprising administering to the subject an effective amount of puromycin or puromycin analog.

8. A method of treating a disease associated with a R183Q or Q209 Lmutation in a subject, the method comprising administering to the subject an effective amount of puromycin or puromycin analog.

9. The method of any of claim 2-3 or 5, wherein the vascular malformation or related condition is a capillary malformation, vascular malformation in the brain, vascular malformation in the eye, or Sturge-Weber syndrome.

10. The method of any of claims 2-9, wherein the puromycin or puromycin analog is administered topically, orally, by injection, or by ocular administration.

11. The method of any one of claims 2-10, wherein the subject comprises a GNAQ polynucleotide or polypeptide having a R183Q or Q209L mutation.

12. The method of any one of claims 2-11, wherein the subject is a human.

13. A composition comprising a puromycin or puromycin analog formulated for topical administration, ocular administration, or administration by injection.

14. The method of any of claims 2-12, comprising administering to the subject an effective amount of the composition of claim 13.

15. A transfected human embryonic kidney (HEK) or endothelial cell, comprising an isolated polynucleotide encoding a GNAQ polypeptide comprising a R183Q mutation.

16. The transfected cell of claim 15, further comprising an isolated polynucleotide encoding a puromycin resistance polypeptide.

17. The transfected cell of any of claims 15-16, wherein the cell is HEK293, HEK293T, EA926, EAhy 926, or HUVEC.

18. The transfected cell of any of claims 15-17, wherein the isolated polynucleotide encoding a GNAQ polypeptide comprising a R183Q mutation is in a lentivirus plasmid.

19. The transfected cell of any of claims 15-18, wherein the cell is stably transfected or transiently transfected with the isolated polynucleotide encoding a GNAQ polypeptide comprising a R183Q mutation.

20. A method of identifying a candidate agent that modulates a GNAQ R183Q or Q209L mutation-associated disease, the method comprising
(a) contacting a cell comprising a GNAQ polynucleotide or polypeptide having a R183Q or Q209L mutation with puromycin and a candidate agent; and
(b) comparing viability of the contacted cell with a reference level of viability, wherein an alteration in viability indicates that the candidate agent modulates the GNAQ R183Q or Q209L mutation-associated disease.

21. A method of identifying a candidate agent that modulates a vascular malformation or related condition, the method comprising
(a) contacting a cell comprising a GNAQ polynucleotide or polypeptide having a R183Q or Q209L mutation with puromycin and a candidate agent; and
(b) comparing viability of the contacted cell with a reference level of viability, wherein an alteration in viability indicates that the candidate agent modulates a vascular malformation or related condition.

22. The method of any of claims 20-21, wherein the cell is a cell of any one of claims 15-19.

23. The method of any of claims 20-22, wherein the alteration in viability is positive or negative.

24. The method of any of claims 21-23, wherein the GNAQ R183Q or Q209L mutation-associated disease or the vascular malformation or related condition is a capillary malformation, vascular malformation in the brain, vascular malformation in the eye, or Sturge-Weber syndrome.

25. A method of identifying an agent that modulates a vascular malformation or related condition, the method comprising
(a) contacting a cell with a candidate agent; and
(b) measuring a level or activity of a ID3, TSC22D3, or TEAD3 polynucleotide or polypeptide,
wherein an alteration in the level or activity of the ID3, TSC22D3, or TEAD3 polynucleotide or polypeptide, indicates that the candidate agent modulates a vascular malformation or related condition.

26. The method of claim 25, wherein the cell comprises a GNAQ polynucleotide or polypeptide having a R183Q or Q209L mutation.

27. The method of claim 26, wherein the cell is a cell of any one of claims 15-19.

28. The method of claim 25, wherein the alteration is an increase or decrease in the level or activity of the ID3, TSC22D3, or TEAD3 polynucleotide or polypeptide.


SEQUENCE LISTING (29 sequences)

SEQ ID NO: 1 - protein, 359 aa, Homo sapiens
MTLESIMACCLSEEAKEARRINDEIERQLRRDKRDARRELKLLLLGTGESGKSTFIKQMRIIHGSGYSDEDKRGFTKLVYQNIFTAMQAMIRAMDTLKIPYKYEHNKAHAQLVREVDVEKVSAFENPYVDAIKSLWNDPGIQECYDRRREYQLSDSTKYYLNDLDRVADPAYLPTQQDVLRVRVPTTGIIEYPFDLQSVIFRMVDVGGQRSERRKWIHCFENVTSIMFLVALSEYDQVLVESDNENRMEESKALFRTIITYPWFQNSSVILFLNKKDLLEEKIMYSHLVDYFPEYDGPQRDAQAAREFILKMFVDLNPDSDKIIYSHFTCATDTENIRFVFAAVKDTILQLNLKEYNLV

SEQ ID NO: 2 - DNA, 6343 bp, Homo sapiens
  source 1..6343: mol_type = other DNA
agactatccgctcccaccgcgcccccggcccacctggtggccccggccctggccgccgcccccgcggcggttcccggagctcgtcccggacgcgcgcccgggcggcgggggctcggcggccaccgctgcctcgggggagcgagggcgggagggtgtgtgtgcgcgctgtgagcagggggtgccggcggggctgcagcggaggcactttggaagaatgactctggagtccatcatggcgtgctgcctgagcgaggaggccaaggaagcccggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgtctttgctgccgtcaaggacaccatcctccagttgaacctgaaggagtacaatctggtctaattgtgcctcctagacacccgccctgcccttccctggtgggctattgaagatacacaagagggactgtatttctgtggaaaacaatttgcataatactaatttattgccgtcctggactctgtgtgagcgtgtccacagagtttgtagtaaatattatgattttatttaaactattcagaggaaaaacagaggatgctgaagtacagtcccagcacatttcctctctatcttttttttaggcaaaaccttgtgactcagtgtattttaaattctcagtcatgcactcacaaagataagacttgtttctttctgtctctctctctttttcttttctatggagcaaaacaaagctgatttcccttttttcttcccccgctaattcatacctccctcctgatgtttttcccaggttacaatggcctttatcctagttccattcttggtcaagtttttctctcaaatgatacagtcaggacacatcgttcgatttaagccatcatcagcttaatttaagtttgtagtttttgctgaaggattatatgtattaatacttacggttttaaatgtgttgctttggatacacacatagtttcttttttaatagaatatactgtcttgtctcactttggactgggacagtggatgcccatctaaaagttaagtgtcatttcttttagatgtttaccttcagccatagcttgattgctcagagaaatatgcagaaggcaggatcaaagacacacaggagtcctttcttttgaaatgccacgtgccattgtctttcctcccttctttgcttctttttcttaccctctctttcaattgcagatgccaaaaaagatgccaacagacactacattaccctaatggctgctacccagaacctttttataggttgttcttaatttttttgttgttgttgttcaagcttttcctttcttttttttcttggtgtttgggccacgattttaaaatgacttttattatgggtatgtgttgccaaagctggctttttgtcaaataaaatgaatacgaacttaaaaaataaaagctggtatcttaaaatgtaagagagtaagactgtgaagcctaaaatgactggctgagaatgaaccagaaatgccatttgccaaacagttgtaactagaaatttgattctcacggtccattcttttctttgtccttaagatgacattgttagtgttcacgtcccatgttcagtgtccaaaccggcaatgtaaaaagtatcctgtgtggtttaacaggaaatctgtttatgtctctttatttgaaaccagttttactctcagtggttctttaagttcaatgaagtctgccaggaacattggttggtagtattattccgacacctttaatttccaaaatctgaagttcctgctagtttaccaccttcatgatcttcttgaactggtaactgattaggttgaacttatggaagatttgtggacttaactcaaaagtaacctctcagtgttctatagaacatgtatttgtgtaactgaacctaccaggagaaatgtttggaattctatatgtgcaatttttcaacaaatgcaaaaaaaatacagcacatgtattgacaagcttctgtcaagcagcttgagttgaaatttgatttaagaaaataaatcatgattgttcaaagctgctgggacgttagaattaggccatgatactggtctcattttaactacagtggtatttggcactagtgtaaacttccatataaatcactcttttggaacaacaaagggggagggagaaaaatcacggcctgttaaatgagtaccaaagccgcccaacagtaatgagatgttctcatccttgattctcccagcctcaaacaacacagcttactttttttttcccttgctcagaaagtacctgtaatttaacaaacagactgcctgtaggtatagtgcaattacaaatgctctaatcattgtacatacatctctcttgatattgcagcatccatactggctttgtaatcattaattttttggcagattgaatgtgctgtattgatatgtatctatgtaattgtattgtatgtctatagctaattcacgttttgaataatgttattttatttacttttttaagagaggagaatgtaaatttgtcagtttatttctgactagggatattttctttccatttagaaaagaagaaaaaaaaaaaaccttactgtcatacagagcggtactagcgtcgtgctgtataaaatcatttgcacattcctgagtagaggtatactgattataagacccaaaggtaatttcatagcaaaatacataaaatcagtcggagcttttatacaaacatggaaaccaactttgtagaacttttgccatttgatctaggattggaatatgagcttttatacaattcatattcttatttggcaaatgcacagtttagtattacctctctgatggcctttactagaaaggcagttttagaagctattgtgatccactaaggaaatgttttaacagctagagaccactgcttgcctgaaagggcgttcttaaatttggtgcagcaaaaaaaaaaaaaaaaaaaaaaaaaattaaacaacaacatttgaaggcctacagtgtgtatagagaaaacctcatcacaagatcataagtgttacagttttagggaatcaagatattctatttaatagagctatagtaaatgtagtcaattaaacctgatctcaaagcttgaagaagctgagcaaaacagggaaagattgttatatttgtctttatgaaattgggatggaatttgctatgcagaattgaggtttgtggcttcgctgttcctgtagggtgcatgacaagatcccttctcttgagaaaggaaaaaattgatcaccctagcagcagtgatgcatagaaacctaattttagccacaccagtcaatcgaagctaaaggattttcttttttgtttcttcggggttttattgaaggggctaggggcgggacgggattcttttcagttttgtataaaaacaaagtttactcatgctttatattatattgtgattgcaagcgttataagcgtgtgccactggcctcctattgttgatgcttaggtaatggaggcctgtggtgagttttatggtgacttgggcatgtcttattcaaaaacaaaaacataaaacacagaaacctttcttcagcataccaaggcaagcagccatttcatgactcacttaacacattgcagtgtaccagtttacagatgatttttccctttttgcgtgacatggcagttctaacccccagagaattccttatttgtaaattggaagtttctactatgccttacagagcttaaattcagaagtttgtgcctcatatctgaaacaaagggaaataacacacccattcaaaagtaaataaatctcctagaagtttttgtttttaacatttccatataaagagctctgttgaatgtcatgaatagactggaaaaaaaaattttaagaacctgcatatgttgtttactagcagatgacaactacaaaaggaatctgaagaacacgtaaaacttgtatttttttttttttggtagattaactagcaggcctattttaaaaaggtaattcagctaaagggcaatttacttttttgtacttcagactatcttgattgtcaaagtgtacgaactgtaattttaaaatttatactgccacatgattgtaaattttagttgtcttaagttaggaattggtgaaaagctatttatgctggatttgggtcaaaatgacttatttgcaaaaaaataaataatgggaagaaagggctgtataatgaaatactgcaagactcacatattggttggaaatttccctcaaatcacctaccgattacccttgatttccctttgttttcagtttctcaaaacgaatgaaatgaaatatagcagaatgttaacccatataaaaataaagtgtacccaaatattgtaatgtatattgctgctcttcttcaaattaaataagggtttaaaaccacttaattggtaatcaacatctcaattgatacaaataaggtgtgcttggtatacattaatattttcttccaaagatatatctttggttagaaacacaaaaaaataaaactagtaatattgtatgtttatctatctctacatatttccagcatatgtagcgttaatagatctgtcctggtaactgtgtctttgggatttcattttggttccatcaaattaggaaaagaaatggcttagttgtatatgattagctagagatttttggagccagacacctgctgtttagtagataacttagtacagaccctaaacttgtcatttgtttttctcacagaatagccatttcctgctgtcttcccaatgatcactgccctttcaataacactcttgcctctagaatcatatgttcaaagtatgaatacacacctagcacatagtaggtgctcaaatattaatttcctccttgccttccttatctaccctgtgtcctccatttccccgtatgattccaacccaatatagcaaatgacatttacatgttatgaaaacatctattgggtaaaatcagatcttggataaagaaattctgacttttatataagcttttggtagacagaaaaaacagaaaggtattcgttggtagaacatttttaagttcaggaaagaaagctggaataatactacgtaactttgtccaggttactttgactgaaacacgtttttggtggatttcttttcctcaaagaactctctaaatgcaactccttgctggattcctcacccatcatcctgttggaaacccttactagacctatgtatttagggagttttgtcagaaaacatttttaacttgcagtatttaaaagaatatttactgttcctaaaatgtcattcaaatgcatgtactgtctattgtttggggatgggaactagttttgcaaaaaacacctaatgttgtataataatgccccaatgatcttgctggttaaaaatacagtatttttggccataa

SEQ ID NO: 3 - protein, 119 aa, Homo sapiens
MKALSPVRGCYEAVCCLSERSLAIARGRGKGPAAEEPLSLLDDMNHCYSRLRELVPGVPRGTQLSQVEILQRVIDYILDLQVVLAEPAPGPPDGPHLPIQTAELTPELVISNDKRSFCH

SEQ ID NO: 4 - DNA, 1252 bp, Homo sapiens
  source 1..1252: mol_type = other DNA
gatctggggtgctgccaggaaaaagcaaattctggaagttaatggttttgagtgatttttaaatccttgctggcggagaggcccgcctctccccggtatcagcgcttcctcattctttgaatccgcggctccgcggtcttcggcgtcagaccagccggaggaagcctgtttgcaatttaagcgggctgtgaacgcccagggccggcgggggcagggccgaggcgggccattttgaataaagaggcgtgccttccaggcaggctctataagtgaccgccgcggcgagcgtgcgcgcgttgcaggtcactgtagcgggacttcttttggttttctttctctttggggcacctctggactcactccccagcatgaaggcgctgagcccggtgcgcggctgctacgaggcggtgtgctgcctgtcggaacgcagtctggccatcgcccggggccgagggaagggcccggcagctgaggagccgctgagcttgctggacgacatgaaccactgctactcccgcctgcgggaactggtacccggagtcccgagaggcactcagcttagccaggtggaaatcctacagcgcgtcatcgactacattctcgacctgcaggtagtcctggccgagccagcccctggaccccctgatggcccccaccttcccatccagacagccgagctcactccggaacttgtcatctccaacgacaaaaggagcttttgccactgactcggccgtgtcctgacacctccagaacgcaggtgctggcgcccgttctgcctgggaccccgggaacctctcctgccggaagccggacggcagggatgggccccaacttcgccctgcccacttgacttcaccaaatcccttcctggagactaaacctggtgctcaggagcgaaggactgtgaacttgtggcctgaagagccagagctagctctggccaccagctgggcgacgtcaccctgctcccaccccacccccaagttctaaggtctcttcagagcgtggaggtgtggaaggagtggctgctctccaaactatgccaaggcggcggcagagctggtcttctggtctccttggagaaaggttctgttgccctgatttatgaactctataatagagtatataggttttgtaccttttttacaggaaggtgactttctgtaacaatgcgatgtatattaaactttttataaaagttaacattttgcataataaacgatttttaaacacttgaaaaaaaaaaaaa

SEQ ID NO: 5 - protein, 199 aa, Streptomyces alboniger
MTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 6 - protein, 435 aa, Homo sapiens
MASNSWNASSSPGEAREDGPEGLDKGLDNDAEGVWSPDIEQSFQEALAIYPPCGRRKIILSDEGKMYGRNELIARYIKLRTGKTRTRKQVSSHIQVLARKKVREYQVGIKAMNLDQVSKDKALQSMASMSSAQIVSASVLQNKFSPPSPLPQAVFSTSSRFWSSPPLLGQQPGPSQDIKPFAQPAYPIQPPLPPTLSSYEPLAPLPSAAASVPVWQDRTIASSRLRLLEYSAFMEVQRDPDTYSKHLFVHIGQTNPAFSDPPLEAVDVRQIYDKFPEKKGGLKELYEKGPPNAFFLVKFWADLNSTIQEGPGAFYGVSSQYSSADSMTISVSTKVCSFGKQVVEKVETEYARLENGRFVYRIHRSPMCEYMINFIHKLKHLPEKYMMNSVLENFTILQVVTSRDSQETLLVIAFVFEVSTSEHGAQHHVYKLVKD

SEQ ID NO: 7 - DNA, 3023 bp, Homo sapiens
  source 1..3023: mol_type = other DNA
tcctcaacacaaactttccgtcccgctcgctccctcctccgcgctcggcgcctcccgctccagcccggctcattccgcacattccggccagccccctccccacgaccccccttccccggccccccttgcggctccctcgggcccggcggagcggcccggccggagcgcccccgcgagctcggaccaggctcagccgcccagtgggctcaggcccagagcccagagcaaccagcacaatagcgtccaacagctggaacgccagcagcagccccggggaggcccgggaggatgggcccgagggcctggacaaggggctggacaacgatgcggagggcgtgtggagcccggacatcgagcagagcttccaggaggccctggccatctacccgccctgcggccggcggaagatcatcctgtcagacgagggcaagatgtacggccgaaatgagttgattgcacgctatattaaactgaggacggggaagactcggacgagaaaacaggtgtccagccacatacaggttctagctcggaagaaggtgcgggagtaccaggttggcatcaaggccatgaacctggaccaggtctccaaggacaaagcccttcagagcatggcgtccatgtcctctgcccagatcgtctctgccagtgtcctgcagaacaagttcagcccaccttcccctctgccccaggccgtcttctccacttcctcgcggttctggagcagcccccctctcctgggacagcagcctggaccctctcaggacatcaagccctttgcacagccagcctaccccatccagccgcccctgccgccgacgctcagcagttatgagcccctggccccgctcccctcagctgctgcctctgtgcctgtgtggcaggaccgtaccattgcctcctcccggctgcggctcctggagtattcagccttcatggaggtgcagcgagaccctgacacgtacagcaaacacctgtttgtgcacatcggccagacgaaccccgccttctcagacccacccctggaggcagtagatgtgcgccagatctatgacaaattccccgagaaaaagggaggattgaaggagctctatgagaaggggccccctaatgccttcttccttgtcaagttctgggccgacctcaacagcaccatccaggagggcccgggagccttctatggggtcagctctcagtacagctctgctgatagcatgaccatcagcgtctccaccaaggtgtgctcctttggcaaacaggtggtagagaaggtggagactgagtatgccaggctggagaacgggcgctttgtgtaccgtatccaccgctcgcccatgtgcgagtacatgatcaacttcatccacaagctgaagcacctgcccgagaagtacatgatgaacagcgtgctggagaacttcaccatcctgcaggtggtcacgagccgggactcccaggagaccctgcttgtcattgcttttgtcttcgaagtctccaccagtgagcacggggcccagcaccatgtctacaagctcgtcaaagactagggtgccctctgcgcctccttaaggatgcagggtgagcatctcctctccacacctgcctggcacccctgggggggtccaggattgaggattcatctacctgccaggcctcaggcccaggacccaggaggcctccccacctaccccagcacacacactccctgccactgttctgcgctttaattgtgggagaagagaggagaggagggctcagcggtggggcagcctgtccggggcgctgacccaccatcaccctgctctgcccagcctcgcgtgacctcagagaggtggggataggggacaccttcagcctccagcatgtgtggccactgtacccccacccacccttgggggagcatgatgggcaggtgagggcaggatggagaccaagggagtcagtgagcagaggccctgggagtgtccggttggggttggactgaggacagaggggcccacacttccttgcccctttgtgtcccaggcctggtgcccagactccttgcatggcttgtgtggtcctcagactccgcacagcgagcgtaggtctctgggtttcagatgaagtgcccaggctccaggaagttgagggacccacaggagaggtgggcagagctggagttctcatccagggctgcttgtccccagagcccaggtttatactacctccctggggcgggggctggccgcagggtaggggagaggctctgcagtgtggagtggagcctcatcgaggggcgctgggttaggggagcacctgtttcagactgggcatgaagaagggagcacagcagctactagaccccattagcacctcattagcccacaagccagccaggggccccaggaagatggggcaccccccagcaccctccagattgagagcaaggtagaggaaggagtcccagcctctgggcagaccagaggcccagagggagagagtagcagaaggcttttgatttttctcttgcctgaggcttgaatctgacaaacccttggtgggcactgctcccttaggttcttccccacctcaatctacctgcctagagtagcagctcccagacccagttctgggactgaaggttaacccttcacctgctgtcccttcttaacacccaggcccccagagccagctgggcctgtccagcagccacctgtgggtatttatgagtttcatatgaagtactgtgccccttcccttcctcatcccgaccctgcccgagcttcctgaaggtcctcactgtttgcatatcgctcaggccacctccaaaccccacctaggttttataatgtatattatatatttttttgtgtatttttaaaatccagctgtgatgggttatatcataaatgcagcttggggttggagcaggggccctcaaaggcccagctcctgctcaaaaaaaaaaaaaaaaaaaaattaaagttatttgtttgtgggtcagtcatgtaaaaaaaaaaaaaaaaaaaaaaaaaaaaaaaaaa

SEQ ID NO: 8 - protein, 200 aa, Homo sapiens
MAQSKLDCRSPVGLDCCNCCLDLAHRSGLQRGSSGENNNPGSPTVSNFRQLQEKLVFENLNTDKLNSIMRQDSLEPVLRDPCYLINEGICNRNIDQTMLSILLFFHSASGASVVAIDNKIEQAMDLVKNHLMYAVREEVEILKEQIRELVEKNSQLERENTLLKTLASPEQLEKFQSCLSPEEPAPESPQVPEAPGGSAV

SEQ ID NO: 9 - DNA, 2223 bp, Homo sapiens
  source 1..2223: mol_type = other DNA
gtcatatcccagtgctgactcccgggcgtgcagaccgctaactagctcactcgctctcagctcctgccaccgctcagccgtcacagcccaggggagcccgagagcctgagagcctgcaaaccggggagggagggagcaaaggagggagggagcaagggcgcgccctggctctccctctgccctgctgcccgcccttcctgcctccacaggcaccctggagtcccctcaggccagctcggtgggcgcgcacctgccagccgcccctgacctcgcaggccaggcgacctccgagcctgagaagatggcccagtccaagctcgattgccgctcacctgtcggcctcgactgctgcaactgctgcctggacctggcccatcggagtgggctccagcgaggcagcagcggggagaacaacaacccgggcagccctacagtgagcaactttcggcagctgcaggaaaagctggtctttgagaacctcaataccgacaagctcaacagcataatgcggcaggattcgctagagccggtgctgcgggacccctgctacctgatcaacgagggcatctgcaaccgcaacatcgaccagaccatgctctccatcctgctcttcttccacagtgcctccggagccagcgtggtggccatagacaacaagatcgaacaggccatggatctggtgaagaatcatctgatgtatgctgtgagagaggaggtggagatcctgaaggagcagatccgagagctggtggagaagaactcccagctagagcgtgagaacaccctgttgaagaccctggcaagcccagagcagctggagaagttccagtcctgtctgagccctgaagagccagctcccgaatccccacaagtgcccgaggcccctggtggttctgcggtgtaagtggctctgtcctcagggtgggcagagccactaaacttgttttacctagttctttccagtttgtttttggctccccaagcatcatctcacgaggagaactttacacctagcacagctggtgccaagagatgtcctaaggacatggccacctgggtccactccagcgacagacccctgacaagagcaggtctctggaggctgagttgcatggggcctagtaacaccaagccagtgagcctctaatgctactgcgccctgggggctcccagggcctgggcaacttagctgcaactggcaaaggagaagggtagtttgaggtgtgacaccagtttgctccagaaagtttaaggggtctgtttctcatctccatggacatcttcaacagcttcacctgacaacgactgttcctatgaagaagccacttgtgttttaagcagaggcaacctctctcttctcctctgtttcgtgaaggcaggggacacagatgggagagattgagccaagtcagccttctgttggttaatatggtataatgcatggctttgtgcacagcccagtgtgggattacagctttgggatgaccgcttacaaagttctgtttggttagtattggcatagtttttctatatagccataaatgcgtatatatacccatagggctagatctgtatcttagtgtagcgatgtatacatatacacatccacctacatgttgaagggcctaaccagccttgggagtattgactggtcccttacctcttatggctaagtctttgactgtgttcatttaccaagttgacccagtttgtcttttaggttaagtaagactcgagagtaaaggcaaggaggggggccagcctctgaatgcggccacggatgccttgctgctgcaaccctttccccagctgtccactgaaacgtgaagtcctgttttgaatgccaaacccaccattcactggtgctgactacatagaatggggttgagagaagatcagtttgggcttcacagtgtcatttgaaaacgttttttgttttgttttgtaattattgtggaaaactttcaagtgaacagaaggatggtgtcctactgtggatgagggatgaacaaggggatggctttgatccaatggagcctgggaggtgtgcccagaaagcttgtctgtagcgggttttgtgagagtgaacactttccactttttgacaccttatcctgatgtatggttccaggatttggattttgattttccaaatgtagcttgaaatttcaataaactttgctctgtttttctaaaaataaaaaaaaaaaaaaaaaaaaaaaa

SEQ ID NO: 10 - DNA, 1050 bp, synthetic construct
  source 1..1050: mol_type = other DNA
nnnnnnnnnnnnnnnnnngagctctctggctaactagagaacccactgcttactggcttatcgaaattaatacgactcactatagggagacccaagctggctagcgtttaaacttaagcttggtaccaccatgactctggagtccatcatggcgtgctgcctgagcgaggaggccaaggaagcccggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgaga

SEQ ID NO: 11 - DNA, 1050 bp, synthetic construct
  source 1..1050: mol_type = other DNA
ggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgtctttgctgccgtcaaggacaccatcctccagttgaacctgaaggagtacaatctggtctaactcgagtctagngnnnnnnnnnnnn

SEQ ID NO: 12 - DNA, 1050 bp, synthetic construct
  source 1..1050: mol_type = other DNA
nnnnnnnnnnnnncttnnncttggtaccnccatgactctggagtccatcatggcgtgctgcctgagcgaggaggccaaggaagcccggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcaagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgt

SEQ ID NO: 13 - DNA, 1050 bp, synthetic construct
  source 1..1050: mol_type = other DNA
gatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcaagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgtctttgctgccgtcaaggacaccatcctccagttgaacctgaaggagtacaatctggtctaacnngannnnnnnnnnnnnnnnnnnnnnnc

SEQ ID NO: 14 - DNA, 1050 bp, synthetic construct
  source 1..1050: mol_type = other DNA
nnnnnnnnnnnnnnnnnnnanctctctggctanctagagaacccactgcttactggcttatcgaaattaatacgactcactatagggagacccaagctggctagcgtttaaacttaagcttggtaccaccatgactctggagtccatcatggcgtgctgcctgagcgaggaggccaaggaagcccggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggcctaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgaga

SEQ ID NO: 15 - DNA, 1050 bp, synthetic construct
  source 1..1050: mol_type = other DNA
ggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggcctaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgtctttgctgccgtcaaggacaccatcctccagttgaacctgaaggagtacaatcnggtctaactcgagtctagngnnccnnnnnnnn

SEQ ID NO: 16 - DNA, 20 bp, synthetic construct
  source 1..20: mol_type = other DNA
aggtcactgtagcggacttc

SEQ ID NO: 17 - DNA, 20 bp, synthetic construct
  source 1..20: mol_type = other DNA
cttcatgctggggagtgagt

SEQ ID NO: 18 - DNA, 20 bp, synthetic construct
  source 1..20: mol_type = other DNA
tctgtttcgtgaaggcaggg

SEQ ID NO: 19 - DNA, 20 bp, synthetic construct
  source 1..20: mol_type = other DNA
tgtaatcccacactgggctg

SEQ ID NO: 20 - DNA, 20 bp, synthetic construct
  source 1..20: mol_type = other DNA
ttgtgtaccgtatccaccgc

SEQ ID NO: 21 - DNA, 20 bp, synthetic construct
  source 1..20: mol_type = other DNA
ttctccagcacgctgttcat

SEQ ID NO: 22 - DNA, 20 bp, synthetic construct
  source 1..20: mol_type = other DNA
gcaccatgtcctggatgtca

SEQ ID NO: 23 - DNA, 20 bp, synthetic construct
  source 1..20: mol_type = other DNA
cctgctggaaaacacacagc

SEQ ID NO: 24 - DNA, 20 bp, synthetic construct
  source 1..20: mol_type = other DNA
tccaactttgactgcgggtc

SEQ ID NO: 25 - DNA, 20 bp, synthetic construct
  source 1..20: mol_type = other DNA
acatctgcccgttctctgat

SEQ ID NO: 26 - DNA, 20 bp, synthetic construct
  source 1..20: mol_type = other DNA
atggaaatccaggcagccaa

SEQ ID NO: 27 - DNA, 22 bp, synthetic construct
  source 1..22: mol_type = other DNA
tgtccataactggagtaaccgc

SEQ ID NO: 28 - DNA, 20 bp, synthetic construct
  source 1..20: mol_type = other DNA
tttgtgctttggggagcaga

SEQ ID NO: 29 - DNA, 20 bp, synthetic construct
  source 1..20: mol_type = other DNA
gctaaggcttccttcagcca